(19) United States
(12) Patent Application Publication
(10) Pub. No.: US 2012/0252793 A1
(43) Pub. Date: Oct. 4, 2012

(54) INTRANASAL BENZODIAZEPINE PHARMACEUTICAL COMPOSITIONS

(76) Inventors: Gary Bream, Cary, NC (US); Moise A. Khayrallah, Morrisville, NC (US); Myoung-Ki Baek, Daejeon (KR); Jae-Hoon Jo, Seoul (KR); Hye-Jin Chang, Daejeon (KR)

(21) Appl. No.: 13/435,837

(22) Filed: Mar. 30, 2012

Related U.S. Application Data
(60) Provisional application No. 61/469,940, filed on Mar. 31, 2011.

(51) Int. Cl. A61K 31/5513; A61P 25/08

(52) U.S. Cl. 514/221

(57) ABSTRACT

The present invention generally relates to intranasal pharmaceutical compositions comprising a benzodiazepine and methods of use thereof that can provide a therapeutic effect without a decrease in blood pressure and/or pulse after administration of the pharmaceutical composition.

### RELATED APPLICATIONS

This application claims the benefit of and priority from'U.S. Provisional Application Ser. No. 61/469,940, filed on Mar. 31, 2011, the disclosure of which is hereby incorporated herein by reference in its entirety.

### FIELD OF THE INVENTION

The present invention generally relates to intranasal pharmaceutical compositions comprising a benzodiazepine and methods of use thereof that can provide a therapeutic effect without a decrease in blood pressure and/or pulse after administration of the pharmaceutical composition.

### BACKGROUND OF THE INVENTION

Acute repetitive seizures (ARS), also referred to as serial seizures, sequential seizures, cluster seizures, or crescendo seizures, are a serious neurological emergency. These episodes of increased seizure activity are associated with significant morbidity and mortality, are debilitating, and can progress to status epilepticus. The goal of treatment is rapid termination of seizure activity because the longer the episode of untreated ARS, the more difficult it is to control and the greater the risk of permanent brain damage.

The current treatment for ARS is intravenous (IV) administration of a benzodiazepine. Intravenous administration, however, requires skilled personnel and transport to a medical facility, which can delay initiation of therapy. Treatment delay is associated with longer seizure duration, greater difficulty in terminating the seizure, prolonged hospitalization, higher mortality, and reduced quality of life.

Most seizure emergencies occur at home, work, or school. Studies over the last fifteen years have demonstrated that out-of-hospital therapy is highly effective and can be safely administered by family members or emergency medical technicians. An alternative therapy for ARS is rectally administered diazepam (Diastat®). However, this treatment remains underutilized. Rectal administration is inconvenient if the seizure occurs away from home and is somewhat difficult to administer and retain during a seizure. In addition, many patients, particularly older children and adults, as well as caregivers object to rectal administration. Accordingly, there is a need for a fast, more convenient, and socially acceptable delivery route for effective management of seizure emergencies.

Intranasal treatment can be easily and safely administered by a patient or a caregiver and can improve the management of seizure emergencies. Intranasal administration of a benzodiazepine can enable treatment to be administered quickly and discreetly, can be easier to administer, and can provide an alternative to rectal administration that may be more attractive to patients and caregivers. However, it can be difficult to develop intranasal formulations that can dissolve sufficient concentrations of benzodiazepine in a practical dosage volume for intranasal administration.

The present invention addresses previous shortcomings in the art by providing intranasal pharmaceutical compositions comprising a benzodiazepine in a sufficient concentration to provide a practical dosage volume. Additionally, these compositions can provide a therapeutic effect without a decrease in blood pressure and/or pulse after administration of the pharmaceutical composition.

### SUMMARY OF THE INVENTION

The present invention provides intranasal pharmaceutical compositions comprising a benzodiazepine that can be suitable for treating seizures (e.g., ARS). The pharmaceutical compositions of the present invention can be advantageous because of the ease, speed, and convenience allowed for by intranasal administration and due to the social acceptance and degree of training required for intranasal administration compared to other forms of administration, such as intravenous and rectal. The pharmaceutical compositions can advantageously further provide a therapeutic effect without a decrease in blood pressure and/or pulse after administration of the pharmaceutical composition. In addition, the pharmaceutical compositions can be beneficial by exhibiting a consistent and/or low coefficient of variation and can provide a benzodiazepine in a sufficient concentration to provide a practical dosage volume for intranasal administration.

In one aspect, the pharmaceutical composition comprises about 1% to about 10% by weight of a benzodiazepine, e.g., diazepam, or a pharmaceutically acceptable salt thereof, about 40% to about 47% by weight of a glycol ether, e.g., diethylene glycol monoethyl ether, and about 45% to about 55% by weight one or more fatty acid esters. In some embodiments of the present invention, the composition further comprises about 0.5% to about 3% by weight water.

Another aspect of the present invention provides pharmaceutical compositions comprising about 1% to about 15% by weight of a benzodiazepine, e.g., diazepam, or a pharmaceutically acceptable salt thereof, about 43% to about 55% by weight of a glycol ether, e.g., diethylene glycol monoethyl ether, about 16% to about 18% by weight one or more fatty acid esters, about 22% to about 25% by weight N-methyl-2-pyrrolidone, about 1% to about 5% by weight water, and about 5% to about 10% by weight ethanol.

A further aspect of the present invention provides pharmaceutical compositions for intranasal administration of a benzodiazepine, comprising a benzodiazepine, e.g., diazepam, or a pharmaceutically acceptable salt thereof, a glycol ether, e.g., diethylene glycol monoethyl ether, and one or more fatty acid esters, wherein upon administration to a human subject, plasma levels of diazepam exhibit a coefficient of variation (CV) of less than about 40%.

Another aspect of the present invention provides methods of preventing a drop in blood pressure and/or pulse in a subject during administration of a benzodiazepine, e.g., diazepam, for treatment of a seizure, comprising intranasally administering a therapeutically effective amount of any of the pharmaceutical compositions of the present invention to a subject in need thereof.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE DRAWINGS

FIG. 1 shows the mean diazepam concentration-time profiles (0-24 h) after administration of, Formula 1 (Treatment A), Formula 2 (Treatment B), and Diastat® (Treatment C).

FIGS. 2A-L show the individual diazepam concentration-time profiles (0-240 h) for each subject enrolled in the study.

FIG. 3A shows the mean nordiazepam concentration-time profiles after administration of DZNS Formula 1 (Treatment A), DZNS Formula 2 (Treatment B), and Diastat® (Treatment C).

FIG. 3B shows the mean oxazepam concentration-time profiles after administration of DZNS Formula 1 (Treatment A), DZNS Formula 2 (Treatment B), and Diastat® (Treatment C).

FIG. 3C shows the mean temazepam concentration-time profiles after administration of DZNS Formula 1 (Treatment A), DZNS Formula 2 (Treatment B), and Diastat® (Treatment C).

FIG. 4 shows the mean change from pre-dose in systolic blood pressure after administration of Diastat®, Formula 1, or Formula 2.

FIG. 5 shows the mean change from pre-dose in diastolic blood pressure after administration of Diastat®, Formula 1, or Formula 2.

FIG. 6 shows the mean change from pre-dose in heart rate after administration of Diastat®, Formula 1, or Formula 2.

FIG. 7 shows the mean change from pre-dose in respirations after administration of Diastat®, Formula 1, or Formula 2.

FIG. 8 shows the mean change from pre-dose in oxygen saturation levels after administration of Diastat®, Formula 1, or Formula 2.

FIG. 9 shows the spray pattern images of DZNS Formula 2 with modified (A) and standard (B) vial holders.

FIG. 10 shows the spray pattern images of DZNS Formula 1 with modified (A) and standard (B) vial holders.

FIG. 11 shows spray pattern images of DZNS Formula 2 with modified (A) and standard (B) vial holders.

FIG. 12 shows spray pattern images of DZNS Formula 1 with modified (A) and standard (B) vial holders.

### DETAILED DESCRIPTION OF THE INVENTION

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention.

As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. For example, features described in relation to one embodiment may also be applicable to and combinable with other embodiments and aspects of the invention.

Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The term “about,” as used herein when referring to a measurable value such as an amount or concentration (e.g., the amount of the benzodiazepine in the pharmaceutical composition) and the like, is meant to encompass variations of 20%, 10%, 5%, 1%, 0.5%, or even 0.1% of the specified amount.

All patents, patent applications and publications referred to herein are incorporated by reference in their entirety. In case of a conflict in terminology, the present specification is controlling.

### I. Pharmaceutical Compositions

The present invention provides intranasal pharmaceutical compositions comprising a benzodiazepine active agent. “Benzodiazepine(s),” as used herein, refers to compounds comprising a benzodiazepine structure and known to be useful or later identified to be useful for the treatment of seizures. Benzodiazepines include, but are not limited to, alprazolam, bromazepam, chlordiazepoxide, clonazepam, clorazepate, diazepam, estazolam, flurazepam, halazepam, ketazolam, lorazepam, midazolam, nitrazepam, oxazepam, prazepam, quazepam, temazepam, triazolam, pharmaceutically acceptable salts thereof, and mixtures thereof. Unless otherwise stated, benzodiazepine as used herein is meant to include all isomeric (e.g., enantiomeric, diastereomeric, and geometric (or conformational)) forms of the structure and mixtures thereof; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of benzodiazepines are within the scope of the invention. Unless otherwise stated, all tautomeric forms, solvates, and hydrates of benzodiazepines are within the scope of the invention. In particular embodiments of the present invention, the benzodiazepine is diazepam or a pharmaceutically acceptable salt thereof.

“Pharmaceutically acceptable salt(s)” as used herein, are salts that retain the desired biological activity of the parent benzodiazepine compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine; and (c) base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

A benzodiazepine can be present in an amount from about 1% to about 20% by weight of the pharmaceutical composition. In some embodiments of the present invention, the benzodiazepine is present in an amount from about 1% to about 15% or from about 1% to about 10% by weight of the pharmaceutical composition. In particular embodiments of the present invention, the benzodiazepine is present in an amount of about 1%, 1.5%, 2%, 2.5%, 3%, 3.75%, 4%, 4.5%, 5%, 5.5%, 6%, 6.25%, 6.75%, 7%, 7.5%, 8%, 8.75%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, or any range therein. In certain embodiments of the present invention, a pharmaceutical composition of the present invention comprises from about 2 mg of a benzodiazepine to about 15 mg of a benzodiazepine per 100 μL of the pharmaceutical composition or any range therein, such as, but not limited to, about 5 mg to about 10 mg of a benzodiazepine per 100 μL of the pharmaceutical composition. In some embodiments of the present invention, a pharmaceutical composition of the present invention comprises about 2, 3, 4, 5, 6, 7, 8, 9, 10, 11, 12, 13, 14, or 15 mg of a benzodiazepine per 100 μL of the pharmaceutical composition. In particular embodiments of the present invention, a pharmaceutical composition of the present invention comprises about 9 mg of a benzodiazepine per 100 μL of the pharmaceutical composition and in certain embodiments, about 10 mg of a benzodiazepine per 100 μL of the pharmaceutical composition.

In one aspect of the present invention, the pharmaceutical composition comprises, consists essentially of, or consists of: (i) a benzodiazepine, (ii) at least one glycol ether, and (iii) at least one fatty acid ester. “Glycol ether” as used herein refers to an aliphatic ether of ethylene glycol or diethylene glycol, wherein the glycol ether comprises R—O—R′ or R—O—R′—O—R, where R is an aliphatic group and R′ is the remaining glycol portion of the compound. When the glycol ether comprises R—O—R′, the glycol portion is —(CH₂)₂—OH or —(CH₂)₂—O—(CH₂)₂—OH, and when the glycol ether comprises R—O—R′—O—R, the glycol portion is —(CH₂)₂— or —(CH₂)₂—O(CH₂)₂—. The aliphatic portion, R, of a glycol ether can be a C₁-C₈ aliphatic group, which can be saturated, unsaturated, straight chain, branched chain, and/or cyclic. Exemplary glycol ethers include, but are not limited to, ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether, diethylene glycol mono-n-butyl ether, and any combination thereof. In some embodiments of the present invention, the at least one glycol ether is diethylene glycol monoethyl ether, such as, e.g., Transcutol® HP commercially available from Gattefossé.

The at least one glycol ether can be present in an amount from about 30% to about 80% by weight of the pharmaceutical composition. In particular embodiments of the present invention, the at least one glycol ether is present in an amount from about 35% to about 60% by weight, about 35% to about 47% by weight, about 37% to about 46% by weight, about 40% to about 47% by weight, about 43% to about 55% by weight, or about 43% to about 50% by weight of the pharmaceutical composition. In certain embodiments of the present invention, the at least one glycol ether is present in an amount of about 30%, 30.5%, 31%, 31.5%, 32%, 32.5%, 33%, 33.5%, 34%, 34.5%, 35%, 35.5%, 36%, 36.5% 37%, 37.5%, 38%, 38.5%, 39%, 39.5%, 40%, 40.5%, 41%, 41.5%, 42%, 42.5%, 43%, 43.5%, 44%, 44.5%, 45%, 45.6%, 45.7%, 45.8%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, 80%, or any range therein. In some embodiments of the present invention, as the amount of the benzodiazepine in the composition increases, the amount of the at least one glycol ether in the composition decreases correspondingly and vice versa.

“Fatty acid ester” as used herein refers to a compound comprising a R—C(O)—O— group, wherein R comprises a C₁-C₂₄ aliphatic group that can be saturated, unsaturated, straight chain, branched chain, cyclic, substituted, and/or unsubstituted. For example, in some embodiments of the present invention, a fatty acid ester may comprise R—C(O)—O—R′, wherein R and R′ each comprise a C₁-C₂₄ aliphatic group that can be the same or different and can be saturated, unsaturated, straight chain, branched chain, cyclic, substituted and/or unsubstituted. In other embodiments of the present invention, a fatty acid ester may comprise a glyceride moiety and 1, 2, or 3 R—C(O)—O— group(s). Exemplary fatty acid esters include, but are not limited to, caprylocaproyl polyoxylglyceride, isopropyl palmitate, oleoyl polyoxylglyceride, sorbitan monolaurate 20, methyl laurate, ethyl laurate, ethyl myristate, ethyl palmitate, ethyl linoleate, propyl isobutylate, isopropyl laurate, isopropyl myristate, polysorbate 20, propylene glycol monocaprylate, and any combination thereof. The at least one fatty acid ester can be present in the composition in an amount from about 5% to about 60% by weight, about 5% to about 29% by weight, about 10% to about 30% by weight, about 16% to about 18% by weight, about 30% to about 60% by weight, about 40% to about 55% by weight, or about 45% to about 55% by weight of the pharmaceutical composition. In particular embodiments, the at least one fatty acid ester is present in an amount of about 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.7%, 17.5%, 18%, 18.5%, 19%, 19.5%, 20%, 20.5%, 21%, 21.5%, 22%, 22.5%, 23%; 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5% 37%, 37.5%, 38%, 38.5%, 39%, 39.5%, 40%, 40.5%, 41%, 41.5%, 42%, 42.5%, 43%, 43.5%, 44%, 44.5%, 45%, 45.6%, 45.7%, 45.8%, 46%, 46.5%, 47%, 47.5%, 48%, 48.45%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, or any range therein.

In some embodiments of the present invention, the at least one fatty acid ester is selected from the group consisting of caprylocaproyl polyoxylglyceride, isopropyl palmitate, sorbitan monolaurate 20, and any combination thereof. In other embodiments of the present invention, the at least one fatty acid ester is selected from the group consisting of caprylocaproyl polyoxylglyceride, oleoyl polyoxylglyceride, sorbitan monolaurate 20, and any combination thereof. In further embodiments of the present invention, the at least one fatty acid ester is selected from the group consisting of methyl laurate, propylene glycol monocaprylate, and any combination thereof.

In certain embodiments of the present invention, caprylocaproyl polyoxylglyceride, such as, e.g., Labrasol® commercially available from Gattefossé, can be present in an amount from about 5% to about 40% by weight, about 5% to about 25% by weight, about 20% to about 38% by weight, or about 26% to about 34% by weight of the pharmaceutical composition. In some embodiments, caprylocaproyl polyoxylglyceride is present in an amount of about 5%, 5.5%, 6%, 6.25%, 6.75%, 7%, 7.5%, 8%, 8.75%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.3%, 30.4%, 30.5%, 31%, 31.5%, 32%, 32.5%, 33%, 33.5%, 34%, 34.5%, 35%, 35.5%, 36%, 36.5% 37%, 37.5%, 38%, 38.5%, 39%, 39.5%, 40%, or any range therein.

Isopropyl palmitate can be present in an amount from about 2% to about 15% by weight or about 5% to about 10% by weight of the pharmaceutical composition. In some embodiments, isopropyl palmitate is present in an amount of about 2%, 2.5%, 3%, 3.75%, 4%, 4.5%, 5%, 5.5%, 6%, 6.25%, 6.75%, 7%, 7.22%, 7.3%, 7.5%, 8%, 8.75%, 9%, 9.5%, 10%, 10.5%, 11%, 11.5%, 12%, 12.5%, 13%, 13.5%, 14%, 14.5%, 15%, or any range therein.

Sorbitan monolaurate 20, such as, e.g., SPAN® 20 commercially available from Sigma-Aldrich®, can be present in an amount from about 1% to about 20% by weight or about 5% to about 15% by weight of the pharmaceutical composition. In some embodiments, sorbitan monolaurate 20 is present in an amount of about 1%, 1.5%, 2%, 2.5%, 3%, 3.75%, 4%, 4.5%, 5%, 5.5%, 6%, 6.25%, 6.75%, 7%, 7.5%, 8%, 8.75%, 9%, 9.5%, 10%, 10.5%, 10.8%, 11%, 11.2%, 11.4%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, or any range therein.

Oleoyl polyoxylglyceride, such as, e.g., Labrafil® commercially available from Gattefossé, can be present in an amount from about 2% to about 15% by weight or about 5% to about 10% by weight of the pharmaceutical composition. In some embodiments, oleoyl polyoxylglyceride is present in an amount of about 2%, 2.5%, 3%, 3.75%, 4%, 4.5%, 5%, 5.5%, 6%, 6.25%, 6.75%, 7%, 7.22%, 7.5%, 8%, 8.75%, 9%, 9.5%, 10%, 10.5%, 11%, 11.5%, 12%, 12.5%, 13%, 13.5%, 14%, 14.5%, 15%, or any range therein.

Methyl laurate can be present in an amount from about 5% to about 15% by weight or about 9% to about 10% by weight of the pharmaceutical composition. In some embodiments, methyl laurate is present in an amount of about 5%, 5.5%, 6%, 6.25%, 6.75%, 7%, 7.5%, 8%, 8.75%, 9%, 9.5%, 10%, 10.5%, 10.8%, 11%, 11.2%, 11.4%, 11.5%, 12%, 12.5%, 13%, 13.5%, 14%, 14.5%, 15%, or any range therein.

Propylene glycol monocaprylate, such as, e.g., Capryol™ 90 commercially available from Gattefossé, can be present in an amount from about 5% to about 15% by weight or about 7% to about 9% by weight of the pharmaceutical composition. In some embodiments, propylene glycol monocaprylate is present in an amount of about 5%, 5.5%, 6%, 6.25%, 6.75%, 7%, 7.5%, 7.6%, 8%, 8.75%, 9%, 9.5%, 10%, 10.5%, 10.8%, 11%, 11.2%, 11.4%, 11.5%, 12%, 12.5%, 13%, 13.5%, 14%, 14.5%, 15%, or any range therein.

Water can optionally be present in the pharmaceutical compositions of the present invention in an amount from about 0% to about 10% by weight of the pharmaceutical compositions. In particular embodiments, water is present in an amount from about 0.5% to about 5% by weight, from about 0.5% to about 3% by weight, or from about 1% to about 5% by weight of the pharmaceutical composition. In certain embodiments, water is present in an amount of about 0%, 0.25%, 0.5%, 0.75%, 0.95%, 1%, 1.5%, 1.9%, 2%, 2.5%, 3%, 3.5%, 4%, 4.5%, 5%, 5.5%, 6%, 6.25%, 6.75%, 7%, 7.5%, 8%, 8.75%, 9%, 9.5%, 10%, or any range therein.

The pharmaceutical compositions of the present invention can optionally comprise an alcohol. Exemplary alcohols include, but are not limited to, methanol, ethanol, n-propanol, isopropyl alcohol, n-butanol, isobutyl alcohol, 2-butanol, and tert-butyl alcohol. In particular embodiments of the present invention, the pharmaceutical composition comprises ethanol. The alcohol can be present in an amount from about 0% to about 10% by weight or from about 5% to about 10% by weight of the pharmaceutical composition. In certain embodiments, alcohol is present in an amount of about 0%, 0.25%, 0.5%, 0.75%, 0.95%, 1%, 1.5%, 1.9%, 2%, 2.5%, 3%, 3.5%, 4%, 4.5%, 5%, 5.5%, 6%, 6.25%, 6.75%, 7%, 7.5%, 7.6%, 8%, 8.75%, 9%, 9.5%, 10%, or any range therein.

N-methyl-2-pyrrolidone, such as, e.g., Pharmasolve® commercially available from International Specialty Products, can optionally be present in the pharmaceutical compositions of the present invention. In some embodiments of the present invention, N-methyl-2-pyrrolidone is present in an amount from about 0% to about 30% by weight, from about 10% to about 30% by weight, from about 20% to about 30% by weight, or from about 22% to about 25% by weight of the pharmaceutical composition. In certain embodiments, N-methyl-2-pyrrolidone is present in an amount of about 0%, 0.25%, 0.5%, 0.75%, 0.95%, 1%, 1.5%, 1.9%, 2%, 2.5%, 3%, 3.5%, 4%, 4.5%, 5%, 5.5%, 6%, 6.25%, 6.75%, 7%, 7.5%, 8%, 8.75%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 22.7%, 23%, 23.5%, 24%, 24.5%, 25%, 25.5%, 26%, 26.5%, 27%, 27.5%, 28%, 28.5%, 29%, 29.5%, 30%, or any range therein.

In one aspect of the present invention, the pharmaceutical composition comprises about 1% to about 10% by weight diazepam or a pharmaceutically acceptable salt thereof, about 40% to about 47% by weight diethylene glycol monoethyl ether, and about 45% to about 55% by weight one or more fatty acid esters. In other embodiments, the pharmaceutical composition additionally comprises about 0.5% to about 3% by weight water.

Another aspect of the present invention provides a pharmaceutical composition that comprises about 1% to about 10% by weight diazepam or a pharmaceutically acceptable salt thereof, about 60% to about 80% by weight diethylene glycol monoethyl ether, about 5% to about 29% by weight one or more fatty acid esters, and about 0.5% to about 3% by weight water. In another aspect of the present invention, the pharmaceutical composition comprises about 1% to about 10% by weight diazepam or a pharmaceutically acceptable salt thereof, about 40% to about 47% by weight diethylene glycol monoethyl ether, about 26% to about 34% by weight caprylocaproyl polyoxylglyceride, about 5% to about 10% by weight isopropyl palmitate, about 5% to about 15% by weight sorbitan monolaurate 20, and about 0.5% to about 3% by weight water. A further aspect of the present invention provides a pharmaceutical composition that comprises about 1% to about 10% by weight diazepam or a pharmaceutically acceptable salt thereof, about 40% to about 47% by weight diethylene glycol monoethyl ether, about 26% to about 34% by weight caprylocaproyl polyoxylglyceride, about 5% to about 10% by weight oleoyl polyoxylglyceride, and about 5% to about 15% by weight sorbitan monolaurate 20.

In a further aspect of the present invention, the pharmaceutical composition comprises about 1% to about 15% by weight diazepam or a pharmaceutically acceptable salt thereof, about 43% to about 55% by weight diethylene glycol monoethyl ether, about 16% to about 18% by weight one or more fatty acid esters, about 22% to about 25% by weight N-methyl-2-pyrrolidone, about 1% to about 5% by weight water, and about 5% to about 10% by weight ethanol.

In another aspect of the present invention, the pharmaceutical composition comprises about 1% to about 15% by weight diazepam or a pharmaceutically acceptable salt thereof, about 43% to about 55% by weight diethylene glycol monoethyl ether, about 9% to about 10% by weight methyl laurate, about 7% to about 9% by weight propylene glycol monocaprylate, about 22% to about 25% by weight N-methyl-2-pyrrolidone, about 1% to about 5% by weight water, and about 5% to about 10% by weight ethanol.

The pharmaceutical compositions can optionally comprise one or more additional components, such as, but not limited to, carriers, excipients, viscosity-increasing agents, preservers, stabilizers, anti-oxidants, binders, disintegrants, humectants, lubricants, colorants, flavoring agents, corrigents, suspend molding agents, emulsifying agents, solubilizers, buffering agents, tonicity agents, detergents, soothing agents, sulfur-containing reducing agents, etc.

The pharmaceutical compositions of the present invention can be formulated for intranasal administration in accordance with conventional techniques. See, e.g., Remington, The Science and Practice of Pharmacy (20th Ed. 2000). For example, the intranasal pharmaceutical compositions of the present invention can be formulated as an aerosol (this term includes both liquid and dry powder aerosols). Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. As another example, the pharmaceutical compositions of the present invention can be formulated as an on-demand dissolvable form, which provides a lyophilized portion of the pharmaceutical composition and a dissolving solution portion of the pharmaceutical composition.

In some embodiments of the present invention, the pharmaceutical composition is in the form of an aqueous suspension, which can be prepared from solutions or suspensions. With respect to solutions or suspensions, dosage forms can be comprised of micelles of lipophilic substances, liposomes (phospholipid vesicles/membranes) and/or a fatty acid (e.g., palmitic acid). In particular embodiments, the pharmaceutical composition is a solution or suspension that is capable of dissolving in the fluid secreted by mucous membranes of the epithelium of the nasal cavity, which can advantageously enhance absorption.

The pharmaceutical composition can be an aqueous solution, a nonaqueous solution or a combination of an aqueous and nonaqueous solution.

Suitable aqueous solutions include but are not limited to aqueous gels, aqueous suspensions, aqueous microsphere suspensions, aqueous microsphere dispersions, aqueous liposomal dispersions, aqueous micelles of liposomes, aqueous microemulsions, and any combination of the foregoing, or any other aqueous solution that can dissolve in the fluid secreted by the mucosal membranes of the nasal cavity. Exemplary nonaqueous solutions include but are not limited to nonaqueous gels, nonaqueous suspensions, nonaqueous microsphere suspensions, nonaqueous microsphere dispersions, nonaqueous liposomal dispersions, nonaqueous emulsions, nonaqueous microemulsions, and any combination of the foregoing, or any other nonaqueous solution that can dissolve or mix in the fluid secreted by the mucosal membranes of the nasal cavity.

Examples of powder formulations include without limitation simple powder mixtures, micronized powders, powder microspheres, coated powder microspheres, liposomal dispersions, and any combination of the foregoing. Powder microspheres can be formed from various polysaccharides and celluloses, which include without limitation starch, methylcellulose, xanthan gum, carboxymethylcellulose, hydroxypropyl cellulose, carbomer, alginate polyvinyl alcohol, acacia, chitosans, and any combination thereof.

In particular embodiments, the composition is one that is at least partially, or even substantially (e.g., at least 80%, 90%, 95% or more) soluble in the fluids that are secreted by the nasal mucosa (e.g., the mucosal membranes that surround the cilia of the olfactory receptor cells of the olfactory epithelium) so as to facilitate absorption. Alternatively or additionally, the composition can be formulated with a carrier and/or other substances that foster dissolution of the agent within nasal secretions, including without limitation fatty acids (e.g., palmitic acid), gangliosides (e.g., GM-1), phospholipids (e.g., phosphatidylserine), and emulsifiers (e.g., polysorbate 80).

Those skilled in the art will appreciate that because the volume of the pharmaceutical composition administered is generally small, nasal secretions may alter the pH of the administered dose since the range of pH in the nasal cavity can be as wide as 5 to 8. Such alterations can affect the concentration of un-ionized drug available for absorption. Accordingly, in representative embodiments, the pharmaceutical composition further comprises a buffer to maintain or regulate pH in situ. Typical buffers include, but are not limited to, acetate, citrate, prolamine, carbonate, and phosphate buffers.

In embodiments of the invention, the pH of the pharmaceutical composition is selected so that the internal environment of the nasal cavity after administration is on the acidic to neutral side, which (1) can provide the active compound in an un-ionized form for absorption, (2) prevents growth of pathogenic bacteria in the nasal passage, which is more likely to occur in an alkaline environment, and (3) reduces the likelihood of irritation of the nasal mucosa.

For liquid and powder sprays or aerosols, the pharmaceutical composition can be formulated to have any suitable and desired particle or droplet size. In illustrative embodiments, the majority and/or the mean size of the particles or droplets range from equal to or greater than about 1, 2.5, 5, 10, 15 or 20 microns and/or equal to or less than about 25, 30, 40, 45, 50, 60, 75, 100, 125, 150, 175, 200, 225, 250, 275, 300, 325, 350, 375, 400, or 425 microns (including all combinations of the foregoing). Representative examples of suitable ranges for the majority and/or mean particle or droplet size include, without limitation, from about 5 to 100 microns, from about 10 to 60 microns, from about 175 to 325 microns, and from about 220 to 300 microns which facilitate the deposition of an effective amount of the active compound in the nasal cavity (e.g., in the upper third of the nasal cavity, the superior meatus, the olfactory region and/or the sinus region to target the olfactory neural pathway). In general, particles or droplets smaller than about 5 microns will be deposited in the trachea or even the lung, whereas particles or droplets that are about 50 microns or larger generally do not reach the nasal cavity and are deposited in the anterior nose.

International patent publication WO 2005/023335 (Kurve Technology, Inc.) describes particles and droplets having a diameter size suitable for the practice of representative embodiments of the present invention. For example, the particles or droplets can have a mean diameter of about 2 to 50 microns, about 5 to 50 microns, about 5 to 40 microns, about 5 to 35 microns, about 5 to 30 microns, about 5 to 20 microns, about 5 to 17 microns, about 5 to 30 microns, about 10 to 25 microns, about 10 to 15 microns, about 11 to 50 microns, about 11 to 30 microns, about 11 to 20 microns, about 11 to 15 microns, about 12 to 17 microns, about 15 to 25 microns, about 15 to 27 microns or about 17 to 23 microns.

In particular embodiments, the particles or droplets have a mean diameter of about 5 to 30 microns, about 10 to 20 microns, about 10 to 17 microns, about 10 to 15 microns, about 12 to 17 microns, about 10 to 15 microns or about 10 to 12 microns.

Further, the particles or droplets can have a mean diameter of about 10 to 20 microns, about 10 to 25 microns, about 10 to 30 microns, or about 15 to 30 microns.

The particles can “substantially” have a mean diameter or size as described herein, i.e., at least about 50%, 60%, 70%, 80%, 90% or 95 or more of the particles are of the indicated diameter or size range.

The composition is optionally delivered as a nebulized or atomized liquid having a droplet size as described above.

In particular embodiments, the pharmaceutical composition is isotonic to slightly hypertonic, e.g., having an osmolarity ranging from about 150 to 550 mOsM. As another particular example, the pharmaceutical composition is isotonic having, e.g., an osmolarity ranging from approximately 150 to 350 mOsM.

According to particular methods of intranasal delivery, it can be desirable to prolong the residence time of the pharmaceutical composition in the nasal cavity (e.g., in the upper third of the nasal cavity, the superior meatus, the olfactory region and/or in the sinus region), for example, to enhance absorption. Thus, the pharmaceutical composition can optionally be formulated with a bioadhesive polymer, a gum (e.g., xanthan gum), chitosan (e.g., highly purified cationic polysaccharide), pectin (or any carbohydrate that thickens like a gel or emulsifies when applied to nasal mucosa), a microsphere (e.g., starch, albumin, dextran, cyclodextrin), gelatin, a liposome, carbamer, polyvinyl alcohol, alginate, acacia, chitosans and/or cellulose (e.g., methyl or propyl; hydroxyl or carboxy; carboxymethyl or hydroxylpropyl), which are agents that enhance residence time in the nasal cavity. As a further approach, increasing the viscosity of the formulation can also provide a means of prolonging contact of the agent with the nasal epithelium. The pharmaceutical composition can be formulated as a nasal emulsion, ointment or gel, which offer advantages for local application because of their viscosity.

Moist and highly vascularized membranes can facilitate rapid absorption; consequently, the pharmaceutical composition can optionally comprise a humectant, particularly in the case of a gel-based composition so as to assure adequate intranasal moisture content. Examples of suitable humectants include but are not limited to glycerin or glycerol, mineral oil, vegetable oil, membrane conditioners, soothing agents, and/or sugar alcohols (e.g., xylitol, sorbitol; and/or mannitol). The concentration of the humectant in the pharmaceutical composition will vary depending upon the agent selected and the formulation.

The pharmaceutical composition can also optionally include an absorption enhancer, such as an agent that inhibits enzyme activity, reduces mucous viscosity or elasticity, decreases mucociliary clearance effects, opens tight junctions, and/or solubilizes the active compound. Chemical enhancers are known in the art and include chelating agents (e.g., EDTA), fatty acids, bile acid salts, surfactants, and/or preservatives. Enhancers for penetration can be particularly useful when formulating compounds that exhibit poor membrane permeability, lack of lipophilicity, and/or are degraded by aminopeptidases. The concentration of the absorption enhancer in the pharmaceutical composition will vary depending upon the agent selected and the formulation.

To extend shelf life, preservatives can optionally be added to the pharmaceutical composition. Suitable preservatives include but are not limited to benzyl alcohol, parabens, thimerosal, chlorobutanol and benzalkonium chloride, and combinations of the foregoing. The concentration of the preservative will vary depending upon the preservative used, the compound being formulated, the formulation, and the like. In representative embodiments, the preservative is present in an amount of about 2% by weight or less.

The pharmaceutical composition can optionally contain an odorant, e.g., as described in EP 0 504 263 B1 to provide a sensation of odor, to aid in inhalation of the composition so as to promote delivery to the olfactory region and/or to trigger transport by the olfactory neurons.

As another option, the composition can comprise a flavoring agent, e.g., to enhance the taste and/or acceptability of the composition to the subject.

### II. Methods of Treatment

A further aspect of the present invention provides pharmaceutical compositions for intranasal administration of a benzodiazepine, such as, for example, diazepam, to a subject. The term “intranasal administration” as used herein, refers to a systemic form of administration of a benzodiazepine, whereby a benzodiazepine is introduced into one or both of the nasal passages of a subject such that the benzodiazepine contacts the nasal mucosa and is absorbed into the systemic circulation. In certain embodiments, a therapeutically effective amount is administered. Intranasal administration of the pharmaceutical compositions of the present invention can comprise a single administration or multiple administrations of the compositions.

The present invention finds use in both veterinary and medical applications. Suitable subjects of the present invention include, but are not limited to mammals. The term “mammal” as used herein includes, but is not limited to, primates (e.g., simians and humans), non-human primates (e.g., monkeys, baboons, chimpanzees, gorillas), bovines, ovines, caprines, ungulates, porcines, equines, felines, canines, lagomorphs, pinnipeds, rodents (e.g., rats, hamsters, and mice), etc. In some embodiments of the present invention, the subject is a human. Human subjects include both males and females and subjects of all ages including neonatal, infant, juvenile, adolescent, adult, and geriatric subjects.

In some embodiments of the present invention, upon intranasal administration to a subject, plasma levels of the benzodiazepine exhibit a coefficient of variation (CV) of less than about 50%, less than about 40%, less than about 30%, or less than about 20%. In particular embodiments, the benzodiazepine is diazepam. “Coefficient of variation” as used herein refers to the ratio of the standard deviation to the mean value for the maximum benzodiazepine concentration in serum or plasma of a subject (Cₘₐₓ) or the area under the curve (AUC) plotting the serum or plasma concentration of the benzodiazepine along the ordinate (Y-axis) against time along the abscissa (X-axis).

The intranasal pharmaceutical compositions of the present invention, in some embodiments, can provide for a greater absorption of the benzodiazepine and/or a greater bioavailability of the benzodiazepine compared to intravenously and/or rectally administered formulations comprising the benzodiazepine.

Another aspect of the present invention is based on the discovery that after intranasal administration of the pharmaceutical composition to a subject, the subject's blood pressure and/or pulse is maintained at a consistent level. “Consistent level” as used herein refers to a measurement or unit of value that remains within about 25% or less of the initial or control value, which is taken prior to the administration of the pharmaceutical composition. “Prior to administration” as used herein refers to less than an hour before administration of the composition, e.g., less than 30 minutes, 15 minutes, 10 minutes, or 5 minutes. In some embodiments of the present invention, the value remains within about 20% or less, about 15% or less, about 10% or less, or about 5% or less of the initial value prior to administration of the pharmaceutical composition. The subject's blood pressure and/or pulse, in some embodiments, can be maintained at a consistent level for at least about fifteen minutes, thirty minutes, one hour, two hours, three hours, five hours, seven hours, ten hours, or more after administration of the composition.

The subject's blood pressure, in some embodiments, remains within about 25/25 mmHg (SBP/DBP) of the subject's blood pressure prior to administration of the composition. In other embodiments, the subject's blood pressure remains within about 20/20 mmHg, about 15/15 mmHg, about 10/10 mmHg, or about 5/5 mmHg (SBP/DBP) of the subject's blood pressure prior to administration of the composition.

The subject's pulse, in some embodiments, remains within 10 beats per minute of the subject's pulse prior to administration of the composition. In other embodiments, the subject's pulse remains within 9 beats per minute, 8 beats per minute, 7 beats per minute, 6 beats per minute, or 5 beats per minute of the subject's pulse prior to administration of the composition.

A further aspect of the present invention provides methods of treating or preventing a seizure in a subject comprising intranasally administering a therapeutically effective amount of a pharmaceutical composition of the present invention to a subject in need thereof. A subject “in need thereof” as used herein refers to a subject that can benefit from the therapeutic and/or prophylactic effects of the pharmaceutical compositions of the present invention. For example the subject may be experiencing a seizure, has experienced a seizure, is exhibiting or has exhibited signs or symptoms that a seizure is about to occur, and/or is in an at-risk population (e.g., the subject may be at-risk for or more susceptible to seizures).

By the term “treat,” “treating,” or “treatment of” (and grammatical variations thereof) it is meant that the severity of the subject's condition is reduced, at least partially improved or ameliorated, and/or that some alleviation, mitigation or decrease in at least one clinical symptom is achieved and/or there is a delay in the progression of the disease or disorder.

The terms “prevent,” “preventing” and “prevention of” (and grammatical variations thereof) refer to reduction and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

As used herein, the term “therapeutically effective amount” refers to an amount of a benzodiazepine that elicits a therapeutically useful response in a subject. Those skilled in the art will appreciate that the therapeutic effects need not be complete or curative, as long as some benefit is provided to the subject.

Seizures that can be treated and/or prevented according to methods of the present invention include, but are not limited to, primary generalized seizures, such as, absence seizures, atypical seizures, myoclonic seizures, atonic seizures, tonic seizures, clonic seizures, tonic-clonic seizures, and grand mal seizures; partial seizures, such as simple partial seizures, complex partial seizures, and secondary generalized seizures; non-epileptic seizures; acute repetitive seizures; and status epilepticus. “Acute repetitive seizures” as used herein refers to a cluster or number of primary generalized and/or partial seizures that occur over a short period of time, e.g., 30 minutes or less, 20 minutes or less, 15 minutes or less, 10 minutes or less, or 5 minutes or less, in which the subject may regain consciousness between seizures. “Status epilepticus” as used herein refers to an epileptic event in which a primary generalized and/or partial seizure lasts longer than about 5 minutes or in which a series of generalized and/or partial seizures occur during a period longer than about 5 minutes without full recovery of consciousness between seizures. Acute repetitive seizures are related to status epilepticus and one may evolve or turn into the other.

Another aspect of the present invention provides methods of preventing a drop in blood pressure and/or a decrease in pulse in a subject during administration of a benzodiazepine, such as, e.g., diazepam, for the treatment of a seizure, comprising intranasally administering a therapeutically effective amount of a pharmaceutical composition of the present invention to a subject in need thereof.

In some embodiments, the pharmaceutical composition is delivered to the upper third of the nasal cavity, to the superior meatus, the olfactory region and/or the sinus region of the nose. The olfactory region is a small area that is typically about 2-10 cm² in man (25 cm² in the cat) located in the upper third of the nasal cavity for deposition and absorption by the olfactory epithelium and subsequent transport by olfactory receptor neurons. Located on the roof of the nasal cavity, in the superior meatus, the olfactory region is desirable for delivery because it is the only known part of the body in which an extension of the CNS comes into contact with the environment (Bois et al., Fundamentals of Otolaryngology, p. 184, W.B. Saunders Co., Phila., 1989).

The compositions of the present invention are administrated in a manner compatible with the dosage formulation in such an amount as will be effective for the desired result. In particular embodiments, the pharmaceutical composition is administered to the subject in a therapeutically effective amount (as described hereinabove). The quantity to be administered depends on a number of factors, such as, e.g., the subject to be treated and the severity of the condition. Precise amounts of active ingredient required to be administered may depend on the judgment of the practitioner. In general, the dose per subject may be 5 μg, 50 μg, or 250 μg, up to 5 mg, 10 mg, 20 mg, or 100 mg, per dose.

Exemplary dosages include from about 0.001, 0.01 or 0.1 to about 1, 5, 10 or 20 mg/dose, e.g., once, twice or three times daily, two to four times weekly, weekly, two to three times monthly or monthly, or as needed by the subject.

The compound can be administered for a sustained period, such as at least about one month, at least about 2 months, at least about 3 months, at least about 6 months, or at least about 12 months or longer (e.g., as a chronic life-long treatment).

Any suitable dosing schedule can be followed. For example, the dosing frequency can be a once weekly dosing. The dosing frequency can be a once daily dosing. The dosing frequency can be more than once weekly dosing. The dosing frequency can be more than once daily dosing, such as any one of 2, 3, 4, 5, or more than 5 daily doses. The dosing frequency can be intermittent (e.g., one daily dosing for 7 days followed by no doses for 7 days, repeated for any 14 day time period, such as 2 months, 4 months, 6 months or more). The dosing frequency can be continuous (e.g., one weekly dosing for continuous weeks).

In other embodiments, the methods of the invention can be carried out on an as-needed basis by self-medication.

Any of the dosing frequencies can be used with any dosage amount. Further, any of the dosing frequencies and/or dosage amounts can be used with any of the pharmaceutical compositions described herein.

The pharmaceutical composition can be delivered in any suitable volume of administration. In representative embodiments of the invention, the administration volume for intranasal delivery ranges from about 25 microliters to 200 microliters or from about 50 to 150 microliters or from about 50, 100, 250 or 500 microliters to about 1, 2, 3, 3.5 or 4 milliliters in a human. Typically, the administration volume is selected to be large enough to allow for the dissolution of an effective amount of the benzodiazepine but sufficiently small to prevent therapeutically significant amounts of the benzodiazepine from escaping from the anterior chamber of the nose and/or draining into the throat, post nasally.

Intranasal administration of the pharmaceutical compositions of the present invention can be achieved by any known method. In particular embodiments, intranasal administration is by inhalation (e.g., using an inhaler, atomizer or nebulizer device), alternatively, by spray, tube, catheter, syringe, dropper, packtail, pipette, pledget, and the like. As a further illustration, the pharmaceutical composition can be administered intranasally as (1) nose drops, (2) powder or liquid sprays or aerosols, (3) liquids or semisolids by syringe, (4) liquids or semisolids by swab, pledget or other similar means of application, (5) a gel, cream or ointment, (6) an infusion, or (7) by injection, or by any means now known or later developed in the art. In particular embodiments, the method of delivery is by nasal drops, spray or aerosol. As used herein, aerosols can be used to deliver powders, liquids or dispersions (solids in liquid).

In representative embodiments, the pharmaceutical formulation is directed upward during administration, so as to enhance delivery to the upper third (e.g., the olfactory epithelium in the olfactory region) and the side walls (e.g., nasal epithelium) of the nasal cavity. Further, orienting the subject's head in a tipped-back position or orienting the subject's body in Mygind's position or the praying-to-Mecca position can be used to facilitate delivery to the olfactory region.

The formulations can be provided in single or multidose form. In the latter case a means of dose metering can be provided. In the case of a dropper or pipette this may be achieved by the patient or caregiver administering an appropriate, predetermined volume of the composition. In the case of a spray this may be achieved, for example, by means of a metering atomising spray pump.

A further aspect of the present invention is an intranasal spray device comprising a pharmaceutical composition of the present invention.

Many devices are known in the art for nasal delivery. Exemplary devices include particle dispersion devices, bidirectional devices, and devices that use chip-based ink-jet technologies. ViaNase (Kurve Technolgies, Inc., USA) uses controlled particle dispersion technology (e.g., an integrated nebulizer and particle dispersion chamber apparatus, for example, as described in International patent publication WO 2005/023335). Optinose and Optimist (OptiNose, AS, Norway) and DirectHaler (Direct-Haler A/S, Denmark) are examples of bidirectional nasal delivery devices. Ink-jet dispensers are described in U.S. Pat. No. 6,325,475 (MicroFab Technologies, Inc., USA) and use microdrops of drugs on a millimeter sized chip. Devices that rely on iontophoresis/phonophoresis/electrotransport are also known, as described in U.S. Pat. No. 6,410,046 (Intrabrain International NV, Curacao, AN). These devices comprise an electrode with an attached drug reservoir that is inserted into the nose. Iontophoresis, electrotransport or phonophoresis with or without chemical permeation enhancers can be used to deliver the drug to the target region (e.g., olfactory). Other commercially available nasal applicators are, for example, the Pfeiffer unit dose and bidose system, the Valois monospray, bidose and monopowder system or the Becton-Dickinson Accuspray™ system. Also suitable are glass or plastic bottles with commercially available metering pump spray heads.

Nasal delivery devices are also described in U.S. Pat. No. 6,715,485 (OptiNose AS); U.S. Pat. No. 6,325,475 (Microfab Technologies, Inc.); U.S. Pat. No. 6,948,492 (University of Kentucky Research Foundation); U.S. Pat. No. 6,244,573 (LyteSyde, LLC); U.S. Pat. No. 6,234,459 (LyteSyde, LLC); U.S. Pat. No. 6,244,573 (LyteSyde, LLC); U.S. Pat. No. 6,113,078 (LyteSyde, LLC); U.S. Pat. No. 6,669,176 (LyteSyde, LLC); U.S. Pat. No. 5,724,965 (Respironics Inc.); and U.S. Patent Publications US2004/0112378 A1; US 2004/0112379 A1; US 2004/0149289 A1; US 2004/0112380 A1; US 2004; 0182388 A1; US 2005/0028812 A1; US 2005/0235992 A1; US 2005/0072430 A1 and US 2005/0061324 A1.

Further, the pharmaceutical compositions of the present invention can optionally be administered in combination with one or more other therapeutic agents, for example, other therapeutic agents useful in the treatment and/or prevention of seizures or side effects associated with seizures. Exemplary therapeutics include, but are not limited to, anti seizure agents, such as for example, carbamazepine, Carbatrol®, Depakene®, Depakote®, Depakote ER®, dilantin, ethosuximide, felbamate, Felbatol®, gabapentin, Gabitril®, Keppra®, Lamictal®, lamotrigine, levetiracetam, luminal, Mysoline®, Neurontin®, oxcarbazepine, phenobarbital, Phenytek®, phenyloin, primidone, Tegretol®, Tegretol XR®, tiagabine, Topamax®, topiramate, Trileptal®, valproic Acid, Zarontin®, Zonegran®, and Zonisamide, anti-depressants such as, for example, amitryptiline, NMDA receptor antagonists, ion channel antagonists, nicotinic receptor agonists, and antiParkinson's agents, such as for example, deprenyl, amantadine, levodopa, and carbidopa. Other therapeutic agents include, without limitation, barbiturates (e.g., phenobarbital and pentobarbital), steroids (e.g., adrenocorticotropic hormones such as tetracosactide acetate), and anticonvulsants (e.g., hydantoins (phenyloin, ethotoin, etc.), oxazolidines (trimethadione, etc.), succinimides (ethosuximide, etc.), phenacemides (phenacemide, acetylpheneturide, etc.), sulfonamides (sulthiame, acetoazolamide, etc.), aminobutyric acids (e.g. gamma-amino-beta-hydroxybutyric acid, etc.), sodium valproate and derivatives (e.g., valproic acid, valpromide, valproate pivoxil, sodium valproate, semi-sodium valproate), carbamazepine, viagabatrine, tiagabine, and amantadine) and/or any other treatment that may be beneficial to the subject.

As used herein, the administration of two or more compounds “in combination” means that the two compounds are administered closely enough in time that the presence of one alters the biological effects of the other. The two compounds may be administered concurrently, in the same or different formulations, or sequentially. Concurrent administration can be carried out by mixing the compounds prior to administration, or by administering the compounds in two different formulations, for example, at the same point in time but at different anatomic sites or using different routes of administration. As used herein, “concurrent” or “concurrently” means sufficiently close in time to produce a combined effect (that is, concurrently can be simultaneously, or it can be two or more events occurring within a short time period before or after each other).

The present invention is explained in greater detail in the following non-limiting Examples.

### EXAMPLES

### Example 1

An Open-Label, Three-Period, Crossover Study to Determine the Relative Bioavailability of Two Formulations of Diazepam Intranasal Spray (DZNS) versus Diazepam Rectal Gel (Diastat®) in Healthy Volunteers

### Study Objectives:

To determine the pharmacokinetics of diazepam following single 10 mg intranasal doses of DZNS Formula 1 and DZNS Formula 2
To assess the relative bioavailability of diazepam following these two formulations compared to a single 10 mg rectal dose of Diastat®
To evaluate the safety and tolerability of two DZNS formulations (DZNS Formula 1 and DZNS Formula 2)

### Study Design:

This was a single-center, open-label, three-period, randomized, crossover study. The study enrolled 12 healthy adult male or non-pregnant, non-breastfeeding female subjects, between 18 and 50 years of age, inclusive, with a screening body weight of 50-90 kg, inclusive. During each dosing period, subjects received one of the following treatments in a randomized order:
Single 10 mg dose of DZNS Formula 1 (See, Table 1, below), administered as one 5 mg spray (1000) in each nostril, given in the morning. (Lot: 2010J128A)
Single 10 mg dose of DZNS Formula 2 (See, Table 2, below), administered as one 5 mg spray (100 μl) in each nostril, given in the morning. (Lot: 2010J118A)
Single 10 mg dose of Diastat®, administered rectally via the Diastat® AcuDial™, given in the morning. (Lot: CEDH; Expiration: 05/2014)

TABLE 1
DZNS Formula 1

| Ingredient (Trade Name) | % wt/wt |
|---|---|
| Diazepam | 5.0 |
| Diethylene glycol monoethyl ether, NF (Transcutol ® HP) | 45.7 |
| Propylene glycol monocaprylate (Capryol ™ 90) | 7.6 |
| Methyl laurate | 9.5 |
| N-methyl-2-pyrrolidone (Pharmasolve ®) | 22.7 |
| Ethanol, NF | 7.6 |
| Purified Water, USP | 1.9 |

TABLE 2
DZNS Formula 2

| Ingredient (Trade Name) | % wt/wt |
|---|---|
| Diazepam | 5.0 |
| Diethylene glycol monoethyl ether, NF (Transcutol ® HP) | 45.60 |
| Isopropyl palmitate, NF | 7.3 |
| Sorbitan monolaurate, NF (SPAN ® 20) | 10.83 |
| Caprylocaproylpolyoxylglyceride, NF (Labrasol ®) | 30.3 |
| Purified Water, USP | 1.0 |

Subjects who prematurely discontinued following the first dose were not replaced. A screening period of up to 21 days preceded initiation of the treatment period. On Day 0 of each dosing period, subjects checked into the research unit a minimum of 10 hours prior to dosing to undergo assessments to confirm continued eligibility. Subjects received their first treatment dose in the morning (Day 1). Study medication was administered by the research staff.
Administration of the rectal dose of Diastat® was done in compliance with the dosing instructions provided in the Diastat® package insert. Subjects receiving a rectal dose remained in a lateral decubitus position (i.e., lying on one side) for 60 minutes post-dosing, after which full ambulatory movement was permitted if the subject was able and was assisted by clinic staff, as needed. Subjects were asked to refrain from a bowel movement for at least 4 hours post-dosing, if possible. Gauze was placed over the subject's anus immediately after the dose was administered and checked by research staff for visual signs of drug leakage at 15 mins, 30 mins, and 1 hour post-dosing. Any observations of leakage were recorded. Fresh gauze replaced the prior gauze at 15 mins and 30 mins. Gauze was removed permanently at 1 hour post-dosing.
Subjects receiving an intranasal dose were asked to gently blow their nose once, immediately prior to administering the first of the two intranasal diazepam sprays (one per nostril). Prior to and following intranasal administration, the subject's nasal mucosa and throat were examined, and any observation of redness, edema, or abnormality or subject report of nasal or pharyngeal discomfort were recorded. Subjects were dosed in a supine position with their head in a neutral position (facing straight upward) and remained in this position for 10 minutes post-dosing.

After placing the subject in a supine position with their head in a neutral position (facing straight upward), the designated research unit staff member performed the following steps:
1. Inserted the nasal spray tip mid-way into the right nostril, keeping the tip pointed centrally toward the back of the nose.
2. Instructed the subject not to attempt aspiration or inhalation of the spray.
3. Using the thumb, firmly depressed the actuator at the base of the nasal spray device.
4. Repeated steps 1 through 3, for delivery of the second spray into the left nostril and then removed the nasal spray tip from the nose.
   The two sprays were administered to the subject within approximately 15 seconds. After remaining in a supine position for 10 minutes post-dosing, the subject was then placed in a sitting position reclined by 45 degrees (without restriction on head position or movement) until 60 minutes post-dosing, after which full ambulatory movement was permitted if the subject was able and was assisted by clinic staff, as needed. Subjects were asked to refrain from blowing their nose for at least 4 hours post-dosing, if possible. Any visual signs of drug leakage from the nostrils were recorded at 15 mins, 30 mins, and 1 hour post-dosing.
   Subjects remained confined to the research unit until after the 24-hour (Day 2) vital sign measurements and blood sample collection, at which time they were to be discharged. Subjects were to return to the clinic for out-patient visits (PK blood sample collection and vital signs) at the following hours post-dose: 48 (Day 3), 96 (Day 5), 144 (Day 7), 192 (Day 9), and 240 (Day 11). A minimum washout period of 14 days separated each dose administration. Study exit procedures were conducted following the last blood draw of the last dosing period.
   Each intranasal formulation was supplied in a 5 ml amber glass, screw-top bottle, labeled with the formulation name, lot number, and storage conditions. Pfeiffer Bidose nasal spray devices were supplied by Aptar Pharma (Congers, N.Y.). The Pfeiffer Bidose device is a single-use nasal spray device capable of only 2 actuations (one spray per nostril). Each Pfeiffer Bidose device was supplied as 4 separate parts: a vial, vial stopper, vial holder, and actuator.
   Prior to dose administration, pharmacy staff at the clinical research unit filled the nasal spray device vials with the appropriate DZNS formulation to be administered to each subject and then assembled the devices according to the procedures provided by Aptar Pharma. After the nasal spray devices were filled and assembled, the pharmacy staff labeled each device with the DZNS formulation it contained, the date filled, and subject number assigned to receive the dose.
   One spray of the device delivered 0.100 mL of the DZNS formulation. Each dose was administered as two sprays (one spray per nostril given within 15 seconds) containing 5 mg of the DZNS formulation; thus, the total intranasal dose delivered per administration was 10 mg.
   Safety: The Investigator assessed safety using the following parameters: physical examinations, vital signs, pulse oximetry, clinical laboratory evaluations, ECGs, subject alertness observations, nasal and pharyngeal irritation/inflammation examinations (for intranasal doses), and reported or observed adverse events. Subjects were monitored for any adverse events from pre-dose until study completion.
   Pharmacokinetic: A total of 19 serial blood samples were to be collected from each subject during each dosing period at the following times: Pre-dose and 8, 15, 30, and 45 minutes post-dose, and 1, 1.5, 2, 3, 4, 6, 9, 12, 24, 48, 96, 144, 192, and 240 hours post-dose. Blood samples were analyzed for plasma concentrations of diazepam and its major metabolites, desmethyldiazepam, oxazepam, and temazepam, using a validated bioanalytical assay. Plasma concentration-time data are summarized by formulation/treatment with descriptive statistics at each scheduled time point. Individual and mean concentration-time profiles are provided for each treatment.
   Individual diazepam concentration data using nominal sampling times were analyzed using noncompartmental methods (Phoenix WinNonlin Version 6.1). The following PK parameters were determined for diazepam: Cₘₐₓ, Tₘₐₓ, Cₗₐₛₜ, Tₗₐₛₜ, λ_{z}, AUCₗₐₛₜ, AUC_{inf}, % AUC extrapolated. “Cₘₐₓ” as used herein refers to the maximum or peak serum or plasma concentration of the benzodiazepine, e.g., diazepam, in the subject after administration of the benzodiazepine or formulation comprising the benzodiazepine. “Tₘₐₓ” as used herein refers to the time it takes for the benzodiazepine to reach Cₘₐₓ. “Cₗₐₛₜ” as used herein refers to the last quantifiable concentration after dosing of the benzodiazepine or formulation comprising the benzodiazepine. “Tₗₐₛₜ” as used herein refers to the time it takes for the benzodiazepine to reach Cₗₐₛₜ. The term “λ_{z}” as used herein refers to the elimination rate constant for the benzodiazepine, e.g., diazepam. The term “VA” as used herein refers to the elimination half life of the benzodiazepine, e.g., diazepam. “AUCₗₐₛₜ” as used herein refers to the area under the concentration-time curve for the benzodiazepine, e.g., diazepam, from 0 hours to Tₗₐₛₜ. “AUC_{inf}” as used herein refers to the area under the concentration-time curve for the benzodiazepine, e.g., diazepam, from 0 hours to infinity. These PK parameters were summarized using descriptive statistics for each formulation, “Fᵣₑₗ” as used herein refers to the relative bioavailability of the benzodiazepine, e.g., diazepam. Relative bioavailability (Fᵣₑₗ) was calculated as the ratio of the AUC_{inf} values for the test formulations to the reference formulation. PK data for the diazepam metabolites were summarized using descriptive statistics and plotted.
   Data from 12 subjects who completed at least one treatment during the study were included in the pharmacokinetic analyses. Data were missing from treatment with Diastat® for Subjects 204 and 206 and from treatment with DZNS Formula 2 for Subject 202. Concentration-time data that were below the limit of quantification (BLQ) were treated as zero (0.00 ng/mL) in the data summarization and descriptive statistics. In the pharmacokinetic analysis, BLQ concentrations were treated as zero from time-zero up to the time at which the first quantifiable concentration was observed; embedded and/or terminal BLQ concentrations were treated as “missing”.

### Results Summary

### Pharmacokinetic Results:

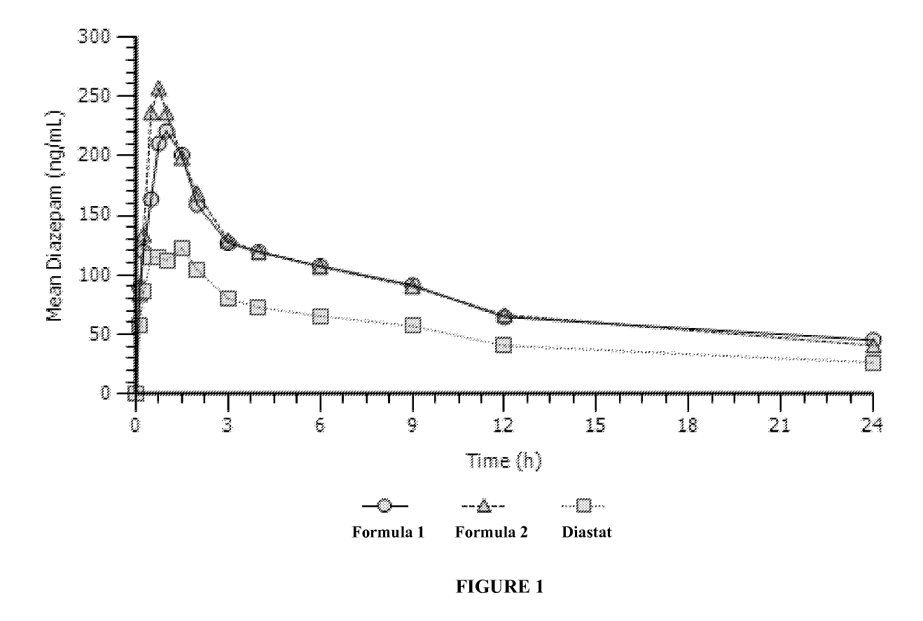

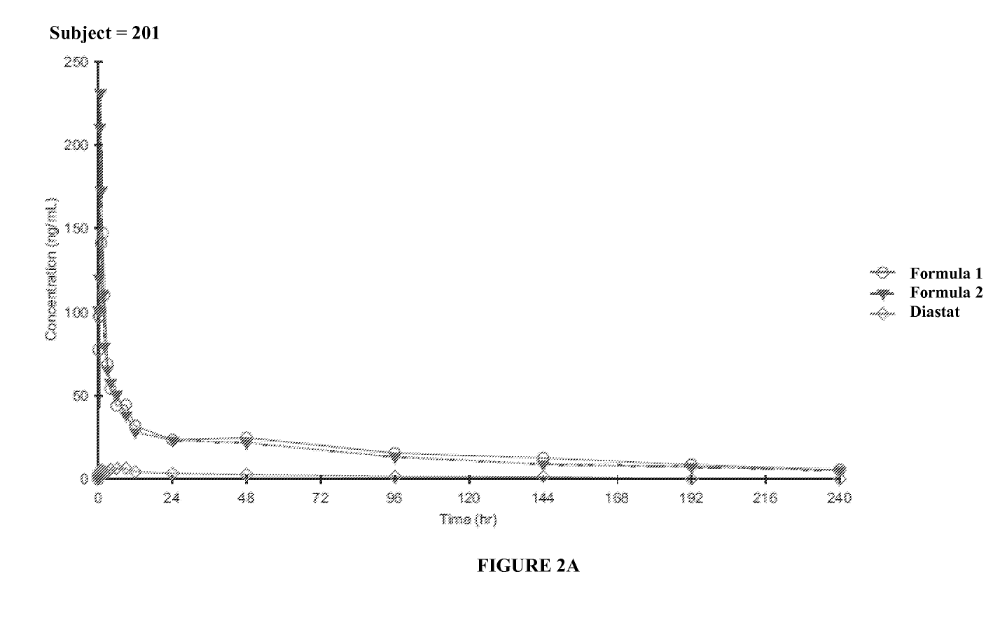

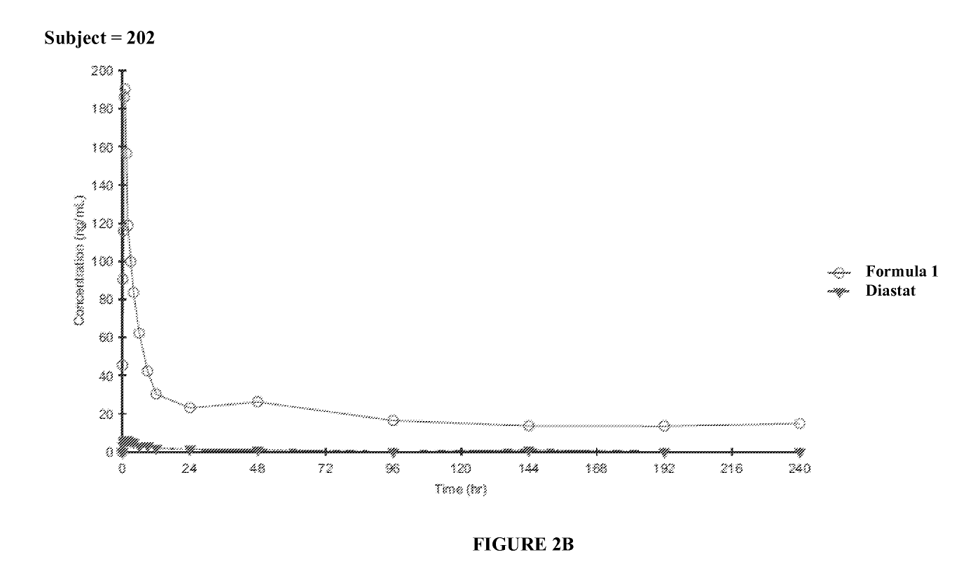

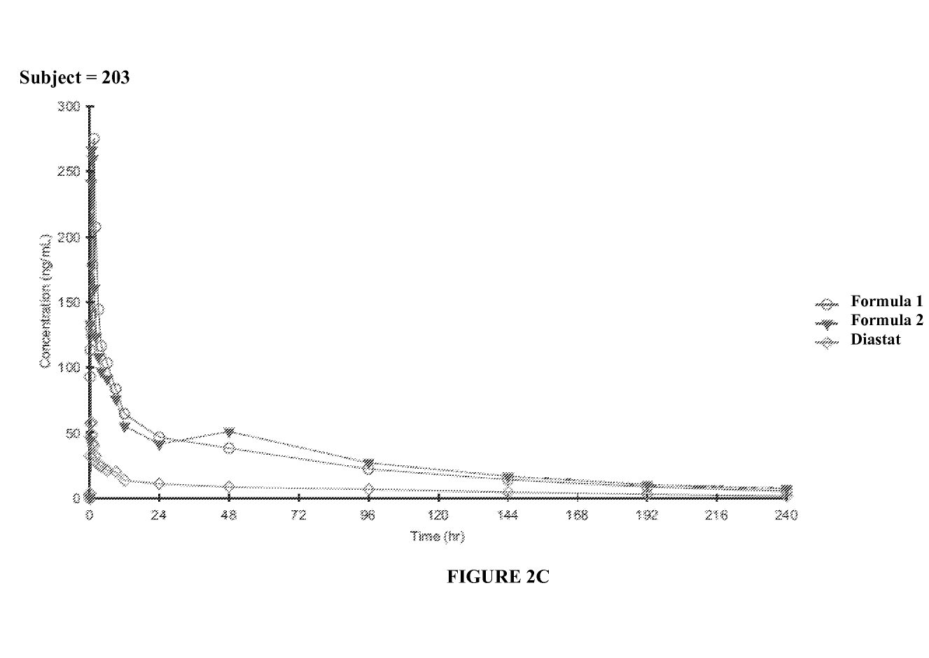

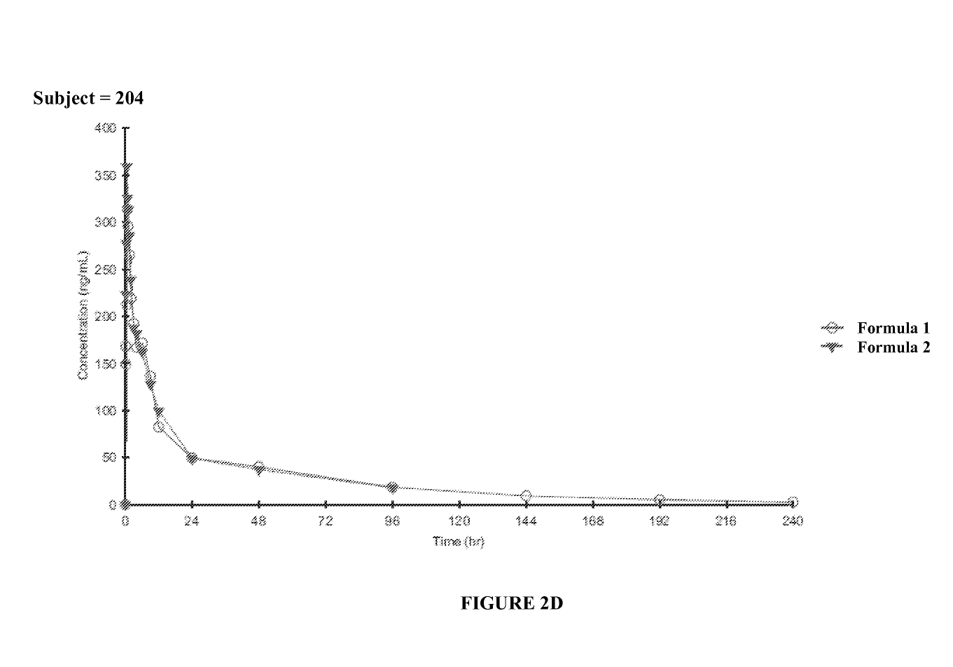

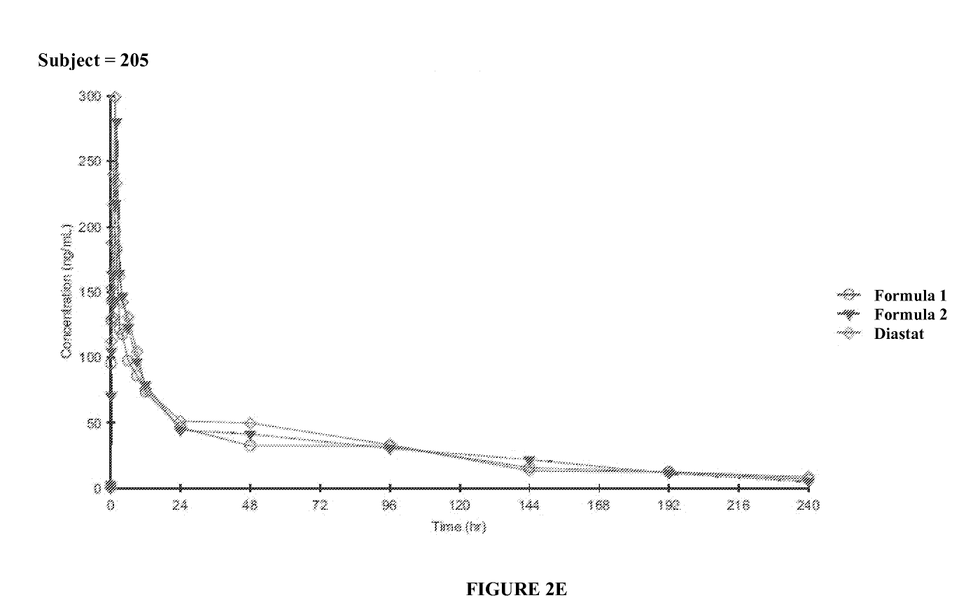

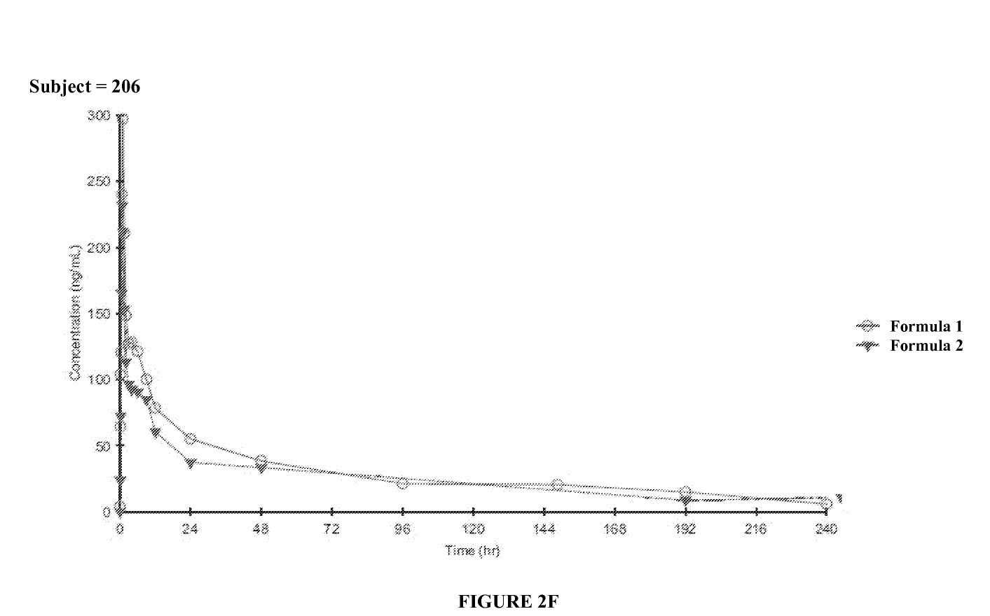

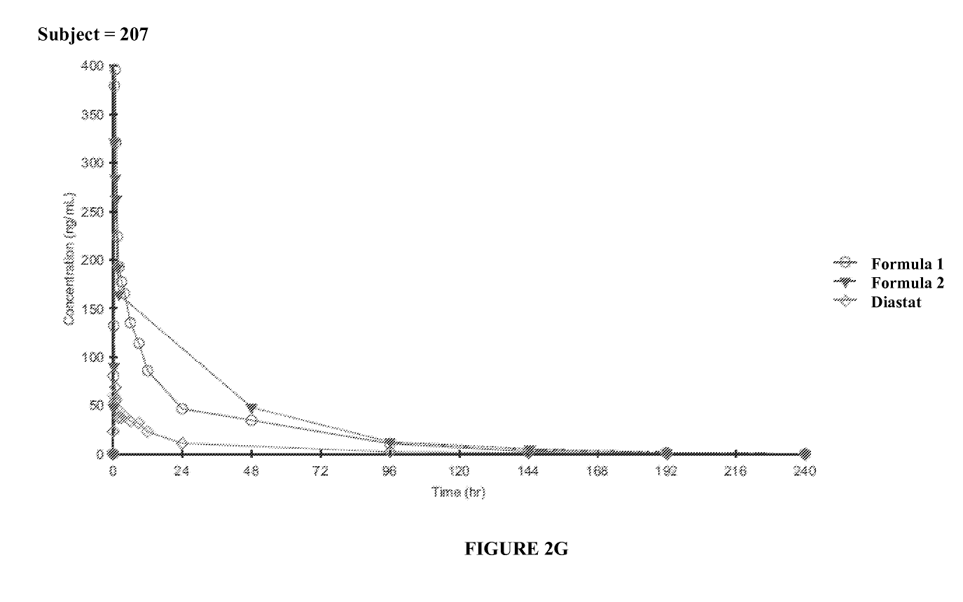

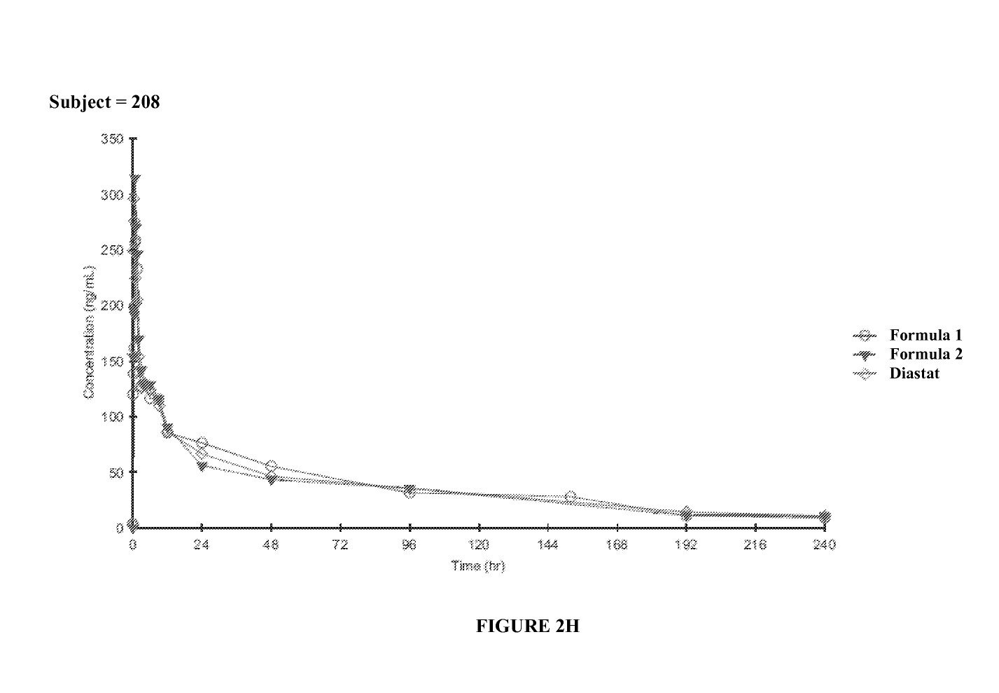

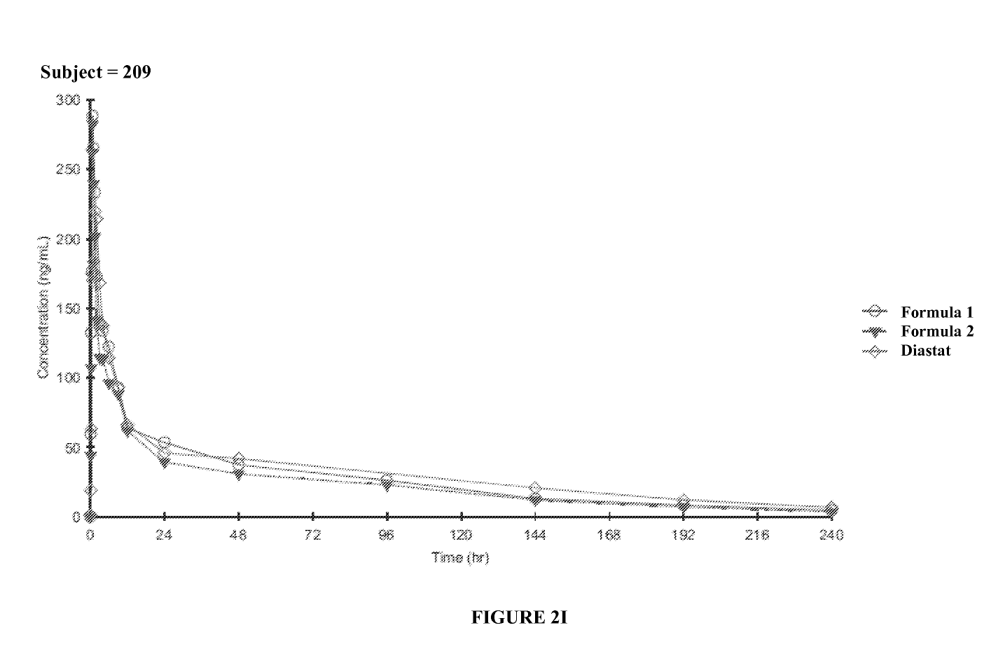

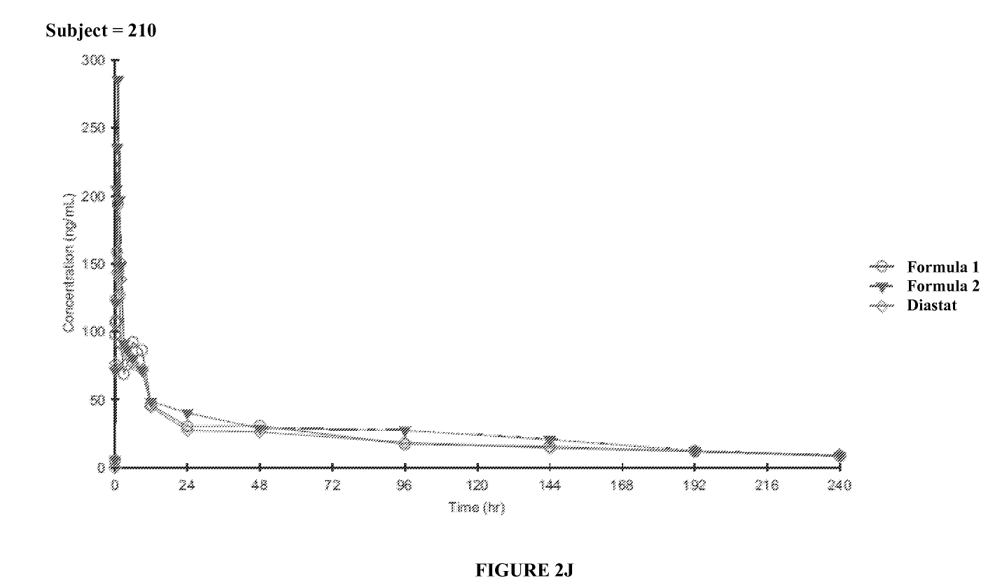

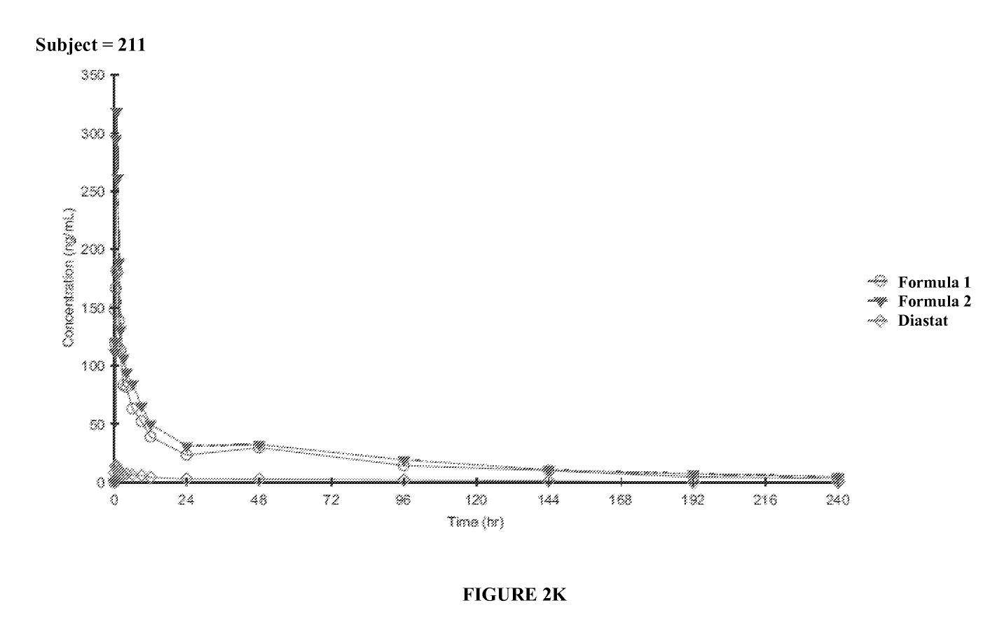

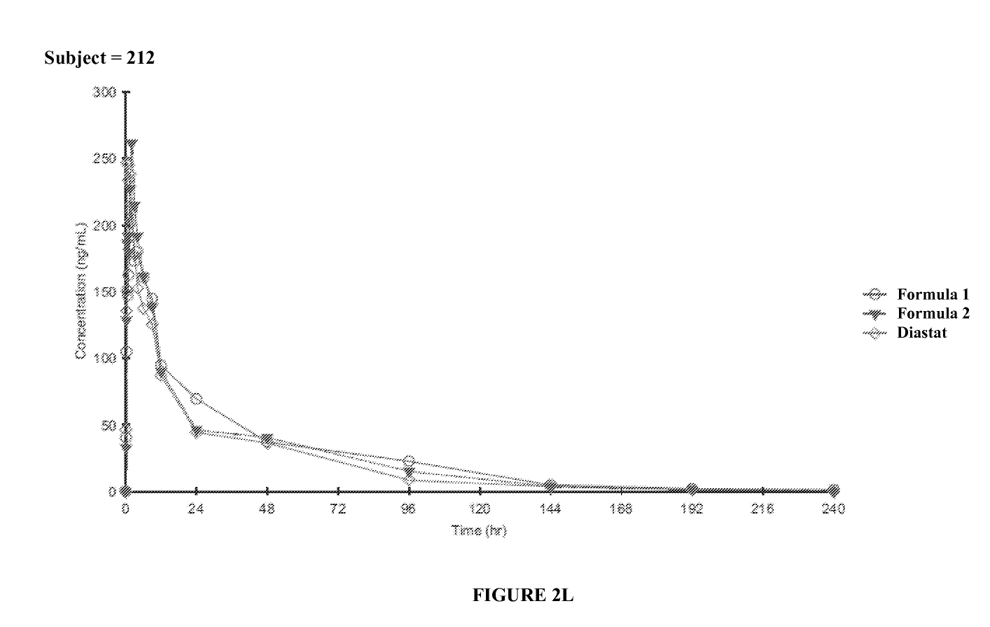

Mean concentration-time data for the 0-24 hour time period are shown in FIG. 1 and individual diazepam concentration-time profiles are displayed in FIG. 2.
Diazepam was rapidly absorbed from all three formulations with the mean peak plasma concentrations occurring 1 to 1.5 hours after dosing. The highest mean plasma concentrations were 221±62.2 ng/mL at 1.00 hr for DZNS Formula 1, 257±56.7 ng/mL at 0.75 hr for DZNS Formula 2, and 122±113 ng/mL at 1.50 hr for Diastat®. Following the peak, the concentrations decayed in a bi-phasic manner with the terminal phase commencing at about 24 hours after dosing. Quantifiable concentrations of diazepam were observed throughout the 240-hr sampling interval for most subjects. Low pre-dose diazepam concentrations were observed in the majority of subjects following Dosing Periods 2, 3, and 4 despite the washout period of 336 hours. The concentrations were very low (average of 1 ng/mL or less) and represented only about 0.5% of the peak concentrations.
Mean diazepam concentrations were considerably lower following the administration of the Diastat® formulation in contrast to either of the intranasal test formulations. Examination of individual subject concentration-time plots indicates that several subjects appeared to have very poor or poor bioavailability of diazepam from the Diastat® formulation. Specifically, subjects 201, 202 and 211 had peak diazepam concentrations of just 6.39, 6.33 and 14.0 ng/mL, respectively, indicating very low bioavailability, and subjects 203 and 207 had concentrations of 58.0 and 63.6 ng/mL, suggesting relatively low bioavailability. In contrast, the remaining 5 subjects who received the Diastat® treatment had peak concentrations ranging from 151 to 299 ng/mL.
As a result of the low concentrations observed in 50% of the Diastat®-treated subjects the variability for the test formulation was much greater than either of the intranasal treatments.
For example, the % CV for the concentrations at 1 hour after dosing is 28.2% for DZNS Formula 1, 22.6% for DZNS Formula 2, and 87.3% for Diastat®.
Although the specific cause of the low concentrations following rectal diazepam is not known, leakage of the formulation was noted in 4 of 5 subjects with low bioavailability, despite careful administration of the drug following the instructions in the labeling. No evidence of leakage was noticed in subjects with good bioavailability.
Results of the pharmacokinetic analysis are shown below in Table 3. For the Diastat® treatment the average Cₘₐₓ was 137 ng/mL and was extremely variable as evidenced by a CV of 88%. The mean Tₘₐₓ was 1.75 hours. The AUC_{inf} averaged 4393 h*ng/mL with a CV of 88%.
In contrast to Diastat®, the Cₘₐₓ for DZNS Formula 1 averaged 246 ng/mL and displayed low variability as evidenced by a CV of 29%. The mean Tₘₐₓ was 1.13 hours. The AUC_{inf} averaged 6969 h*ng/mL with a CV of 24%.
For DZNS Formula 2, Cₘₐₓ averaged 287 ng/mL with a CV of 14%. The mean Tₘₐₓ was 0.95 hour. AUC_{inf} averaged 6918 h*ng/mL with a CV of 21%.

TABLE 3
Summary of Pharmacokinetic Parameters for Diazepam Following Administration of Diastat ®, DZNS Formula 1, or DZNS Formula 2

| | Treatment A: | | | | Treatment B: | | | | Treatment C: | | | |
|---|---|---|---|---|---|---|---|---|---|---|---|---|
| | DZNS Formula 1 | | | | DZNS Formula 2 | | | | Reference Product Diastat ® | | | |
| | 10 mg Intranasal | | | | 10 mg Intranasal | | | | 10 mg Rectal Gel | | | |
| Parameter | n | Mean | SD | CV % | n | Mean | SD | CV % | n | Mean | SD | CV % |
| Tₘₐₓ (hr) | 12 | 1.13 | 0.41 | 36.08 | 11 | 0.95 | 0.53 | 55.95 | 10 | 1.75 | 2.60 | 148.46 |
| Cₘₐₓ (ng/mL) | 12 | 246 | 71.2 | 28.98 | 11 | 287 | 39.2 | 13.67 | 10 | 137 | 121 | 88.25 |
| AUCₗₐₛₜ (hr * ng/mL) | 12 | 6034 | 1423 | 23.58 | 11 | 6196 | 1313 | 21.19 | 10 | 3797 | 3444 | 90.70 |
| AUC_{inf} (hr * ng/mL) | 12 | 6869 | 1663 | 24.21 | 11 | 6918 | 1436 | 20.76 | 10 | 4393 | 3878 | 88.29 |
| AUC_{Extrap} (%) | 12 | 10.90 | 12.77 | 117.24 | 11 | 10.36 | 6.84 | 65.97 | 10 | 20.51 | 20.03 | 97.68 |
| λ_{z} (hr⁻¹) | 12 | 0.0116 | 0.0054 | 46.94 | 11 | 0.0126 | 0.0063 | 49.57 | 10 | 0.0099 | 0.0055 | 55.42 |
| T_{1/2} (hr) | 12 | 75.57 | 46.77 | 1.88 | 11 | 65.52 | 24.69 | 37.68 | 10 | 99.60 | 76.67 | 76.98 |
| Tₗₐₛₜ (hr) | 12 | 236.00 | 13.86 | 5.87 | 11 | 218.63 | 45.07 | 20.61 | 10 | 196.80 | 47.73 | 24.25 |
| Cₗₐₛₜ (ng/mL) | 12 | 5.71 | 3.80 | 66.64 | 11 | 7.05 | 4.88 | 69.15 | 10 | 4.31 | 4.05 | 93.97 |
| CL/F (L/hr) | 12 | 1.539 | 0.3881 | 25.22 | 11 | .508 | 0.3403 | 22.57 | 10 | 7.474 | 7.742 | 103.59 |
| Vz/F (L) | 12 | 158.3 | 76.23 | 48.14 | 11 | 143.5 | 71.55 | 49.86 | 10 | 1345 | 2116 | 157.37 |
| MRT (hr) | 12 | 102.42 | 70.59 | 68.92 | 11 | 86.55 | 33.69 | 38.92 | 10 | 29.74 | 107.45 | 82.82 |

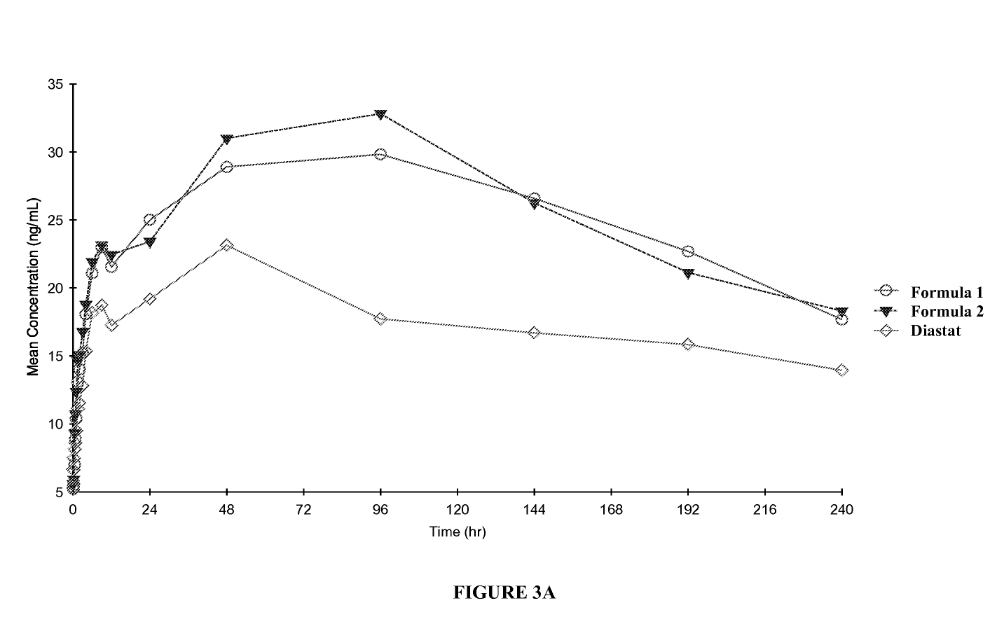

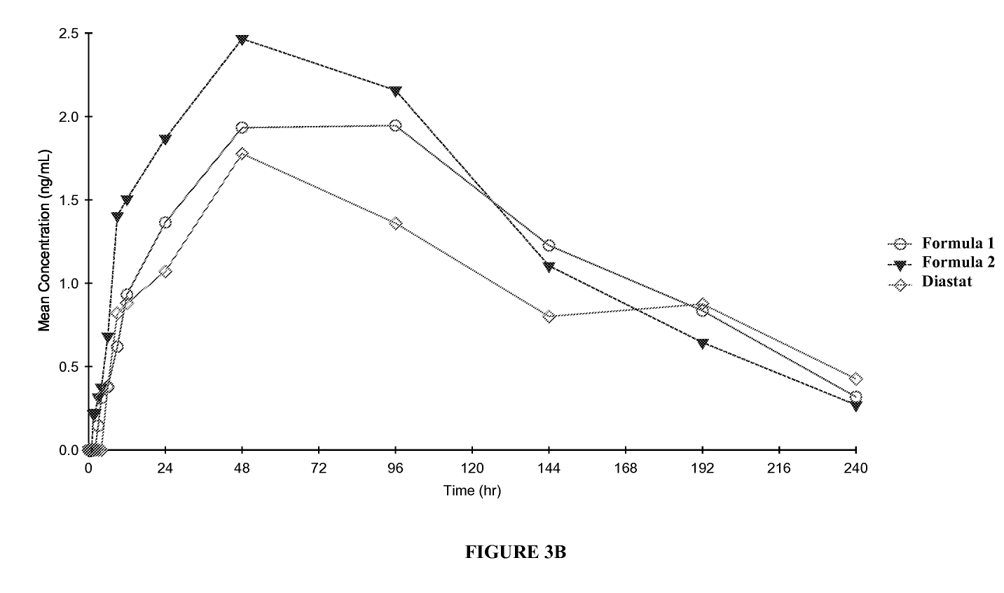

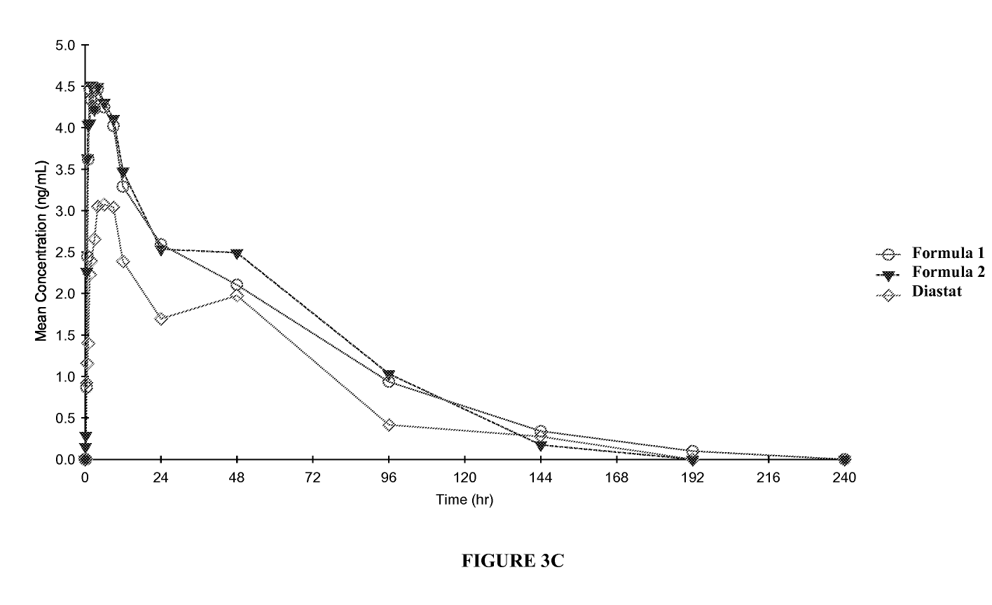

Mean concentration-time profiles for diazepam, N-desmethyldiazepam, oxazepam, and temazepam are plotted by treatment on semi-log axes in FIG. 3. Concentrations of the metabolites display similar profiles for each of the 3 treatments. The Cₘₐₓ and AUC_{inf} ratios calculated for the diazepam metabolites and parent diazepam (metabolite/diazepam) showed that nordiazepam was the most abundant metabolite of diazepam compared to the other 2 metabolites (oxazepam and temazepam). AUC_{inf} ratios for nordiazepam were approximately 2.09, 2.02, and 3.00, respectively, for DZNS Formula 1, DZNS Formula 2, and Diastat®. AUC_{inf} ratios for the other 2 metabolites, oxazepam and temazepam, ranged from approximately 0.05 to 0.21, indicating they are minor metabolites of diazepam following both intranasal and rectal administration.

### Safety Results:

A total of 46 adverse events (AEs) were reported over the course of the study (Table 4). Of the 46 AEs, 41 were mild, 4 were moderate (dizziness for 30 minutes after Treatment B [DZNS Formula 2], beginning about 20 hours after dosing; euphoria and somnolence in one subject for 6 hours after Treatment A [DZNS Formula 1]; and toothache after Treatment A), and 1 was severe (serious AE of trauma with fracture of femur 6 days after Treatment B). Thirty-nine (39) of the AEs were considered by the investigator to be probably related and 7 were considered probably not related to the study drug. There was one SAE due to trauma with fracture of the left femur as the result of a motor vehicle accident, which occurred 6 days after receiving Treatment B. The Investigator judged the SAE to be severe and probably not related to the study drug.

The most commonly reported post-dose AEs were somnolence (n=7; 3 following Treatment A, 2 following Treatment B, and 2 following Treatment C [Diastat®]), throat irritation (n=7; 3 following Treatment A and 4 following Treatment B), and dysgeusia (n=6; 2 following Treatment A and 4 following Treatment B).

TABLE 4
Adverse events after Treatment A (DZNS Formula 1),
Treatment B (DZNS Formula 2), or Treatment C (Diastat ®).

| | Treatment A | | Treatment B | | Treatment C | |
|---|---|---|---|---|---|---|
| | (N = 12) | | (N = 11) | | (N = 10) | |
| Number of | 18 | | 22 | | 6 | |
| Treatment- | | | | | | |
| Emergent | | | | | | |
| Adverse Events | | | | | | |
| Reported | | | | | | |
| Number of | 10 (83%) | | 8 (73%) | | 6 (60%) | |
| Subjects | | | | | | |
| Reporting One | | | | | | |
| or More Events | | | | | | |
| (Percent of | | | | | | |
| Subjects) | | | | | | |
| Adverse Event | Subject | Event | Subject | Event | Subject | Event |
| Bad taste in | 1 (8%) | 1 (6%) | 0 (0%) | 0 (0%) | 0 (0%) | 0 (0%) |
| mouth | | | | | | |
| Burning | 0 (0%) | 0 (0%) | 1 (9%) | 1 (5%) | 0 (0%) | 0 (0%) |
| sensation in | | | | | | |
| throat | | | | | | |
| Dizziness | 0 (0%) | 0 (0%) | 2 (18%) | 2 (9%) | 0 (0%) | 0 (0%) |
| Drowsiness | 0 (0%) | 0 (0%) | 0 (0%) | 0 (0%) | 1 (10%) | 1 (17%) |
| Dry sensation | 1 (8%) | 1 (6%) | 0 (0%) | 0 (0%) | 0 (0%) | 0 (0%) |
| in nose and | | | | | | |
| throat | | | | | | |
| Dysgeusia | 2 (17%) | 2 (11%) | 4 (36%) | 4 (18%) | 0 (0%) | 0 (0%) |
| Erythema left | 0 (0%) | 0 (0%) | 0 (0%) | 0 (0%) | 1 (10%) | 1 (17%) |
| nares | | | | | | |
| Erythema of | 1 (8%) | 1 (6%) | 0 (0%) | 0 (0%) | 0 (0%) | 0 (0%) |
| intranasal | | | | | | |
| mucosa | | | | | | |
| Erythma | 1 (8%) | 1 (6%) | 0 (0%) | 0 (0%) | 0 (0%) | 0 (0%) |
| posterior | | | | | | |
| pharynx | | | | | | |
| Euphoric | 1 (8%) | 1 (6%) | 0 (0%) | 0 (0%) | 0 (0%) | 0 (0%) |
| Fracture, | 0 (0%) | 0 (0%) | 1 (9%) | 1 (5%) | 0 (0%) | 0 (0%) |
| trauma left | | | | | | |
| femur | | | | | | |
| Headache | 0 (0%) | 0 (0%) | 1 (9%) | 1 (5%) | 0 (0%) | 0 (0%) |
| Intermittent | 0 (0%) | 0 (0%) | 1 (9%) | 1 (5%) | 0 (0%) | 0 (0%) |
| cough | | | | | | |
| Intranasal | 1 (8%) | 1 (6%) | 1 (9%) | 1 (5%) | 0 (0%) | 0 (0%) |
| burning | | | | | | |
| sensation | | | | | | |
| Nasal | 1 (8%) | 1 (6%) | 0 (0%) | 0 (0%) | 2 (20%) | 2 (33%) |
| congestion | | | | | | |
| Nasal irritation | 0 (0%) | 0 (0%) | 1 (9%) | 1 (5%) | 0 (0%) | 0 (0%) |
| Nausea | 0 (0%) | 0 (0%) | 1 (9%) | 1 (5%) | 0 (0%) | 0 (0%) |
| Oily skin on | 0 (0%) | 0 (0%) | 1 (9%) | 1 (5%) | 0 (0%) | 0 (0%) |
| face | | | | | | |
| Rhinitis | 1 (8%) | 1 (6%) | 0 (0%) | 0 (0%) | 0 (0%) | 0 (0%) |
| Right elbow | 0 (0%) | 0 (0%) | 1 (9%) | 1 (5%) | 0 (0%) | 0 (0%) |
| pain | | | | | | |
| Somnolence | 3 (25%) | 3 (17%) | 2 (18%) | 2 (9%) | 2 (20%) | 2 (33%) |
| Throat | 3 (25%) | 3 (17%) | 4 (36%) | 4 (18%) | 0 (0%) | 0 (0%) |
| irritation | | | | | | |
| Toothache | 2 (17%) | 2 (11%) | 0 (0%) | 0 (0%) | 0 (0%) | 0 (0%) |
| Watery eyes | 0 (0%) | 0 (0%) | 1 (9%) | 1 (5%) | 0 (0%) | 0 (0%) |

Percentages of subjects (Incidence of AE) are based on the number of subject exposure to each study drug.
Percentages of events are based on the number of events reported.

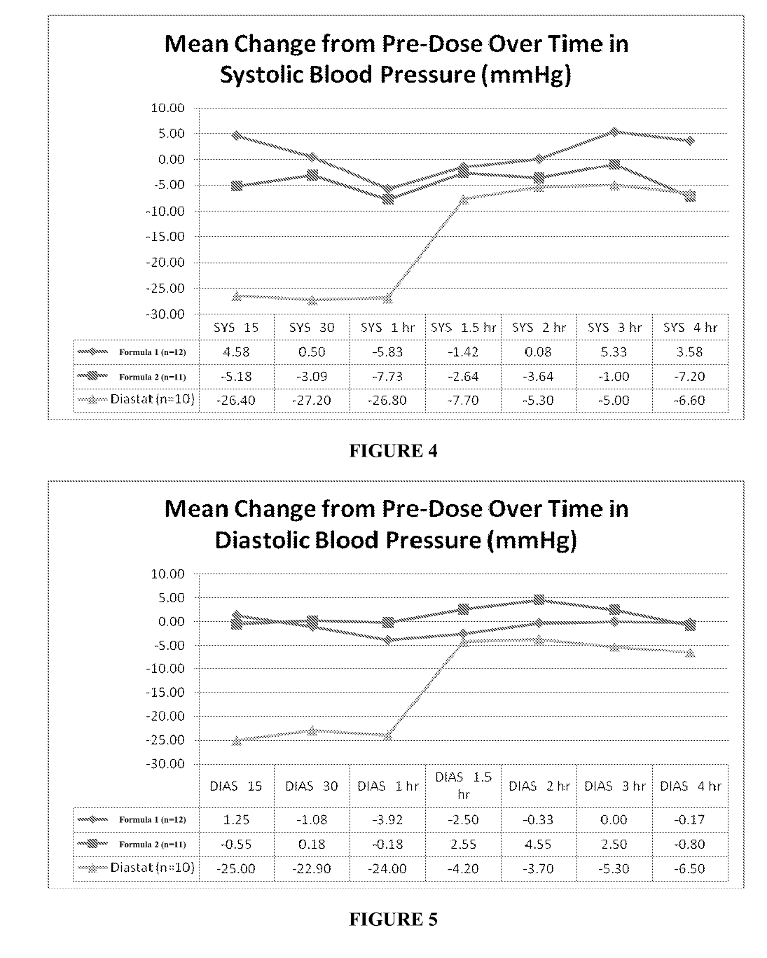

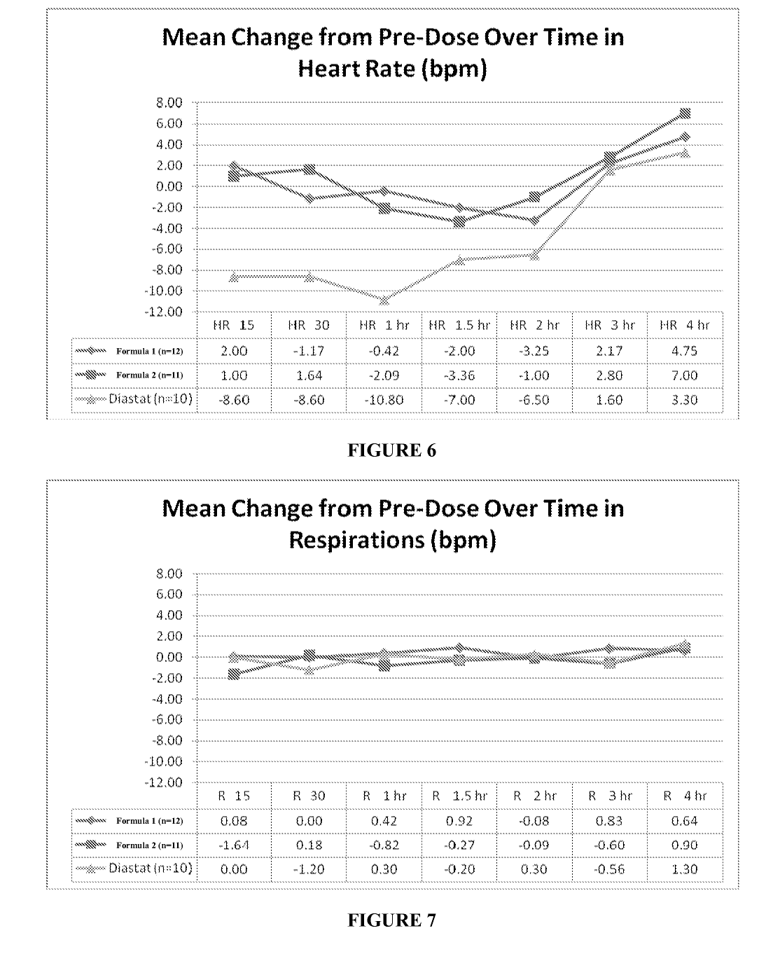

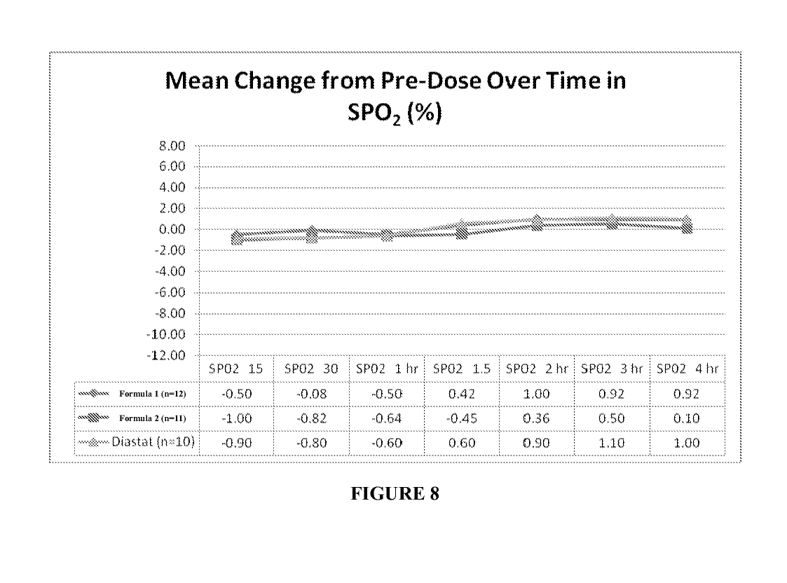

Adverse events reflecting local effects of the intranasal formulation such as throat irritation or dysgeusia, (occurring in 17 to 36% of subjects receiving these formulations) and, less commonly, burning sensation in nose or throat, bad taste in mouth, and signs or symptom of nasal irritation, occurred with about equal frequency in the two nasal formulations but rarely with the rectal formulation. All of these AEs were mild and resolved within 3 hours. AEs reflecting central effects of diazepam, such as somnolence or drowsiness, occurred with about equal frequency in the three treatment groups (18 to 30% of subjects administered each formulation reported one of these two AEs). These AEs were also mild but more variable in duration, usually lasting a few hours.
Nasal and pharyngeal irritation/inflammation assessments following dosing with the intranasal formulations documented signs or symptoms in six subjects which were usually mild, occurred during the first hour after dosing and lasted less than an hour. One subject developed signs of nasal irritation beginning 24 hours after dosing which lasted about one day.
Mean vital sign values at pre-dose (immediately prior to dose administration) for each treatment group are provided below in Table 5. The mean change from pre-dose for each vital sign measurement through 4 hours postdose are displayed in FIGS. 4-8.

TABLE 5
Mean Vital Sign Values at Pre-Dose

| | | MEAN VITAL SIGN VALUES AT PRE-DOSE | | | | |
|---|---|---|---|---|---|---|
| | | Systolic | Diastolic | | | |
| | | Blood | Blood | Heart | | |
| Treatment | | Pressure | Pressure | Rate | Respirations | SP02 |
| Group | N | (mmHg) | (mmHg) | (bpm) | (bpm) | (%) |
| DZNS | 12 | 115.0 | 71.3 | 70.5 | 14.1 | 98.3 |
| Formula 1 | | | | | | |
| DZNS | 11 | 119.9 | 69.8 | 70.5 | 14.6 | 98.8 |
| Formula 2 | | | | | | |
| Diastat ® | 10 | 125.3 | 75.8 | 73.1 | 14.5 | 98.4 |

Following Diastat® AcuDial administration, mean systolic and diastolic blood pressures decreased by 22 to 26 mmHg and heart rate decreased by 9-10 bpm through 1 hour post-dose (FIGS. 2-4). Individual subject changes ranged from −1 to −41 mmHg for systolic blood pressure and −8 to −33 mmHg for diastolic blood pressure over the first hour after dosing. Individual changes for heart rate ranged from +4 to −24 bpm over the same 1 hour interval. No AEs were reported in relation to these changes in vital signs. By comparison no significant changes from pre-dose were observed in mean blood pressure or heart rate following administration of either intranasal formulation. No meaningful changes from pre-dose were seen in respirations or oxygen saturation levels following administration of all three treatments.

Because the effect of rectally delivered diazepam on blood pressure and heart rate observed in this study did not clearly correlate to systemic blood levels of diazepam, it is not clear whether this effect is related to some interaction between the route of delivery and diazepam or a result of the intra-rectal method of delivery itself.

Individual vital sign values for systolic blood pressure, diastolic blood pressure, and heart rate at pre-dose (immediately prior to dose administration) and the 24 hour period following administration of the treatment, for each treatment group (DZNS Formula 1, DZNS Formula 2, or Diastat®) were taken.

### Conclusions:

Diazepam maximum exposure, based on ln(Cₘₐₓ and total systemic exposure, based on ln(AUCₗₐₛₜ) and ln(AUC_{inf}), were substantially higher following administration of the intranasal test formulations (DZNS Formula 1 and DZNS Formula 2) compared to the reference product, Diastat®. Diazepam pharmacokinetic parameter values were comparable for the two intranasal DZNS test formulations.
Overall, the safety profiles of the three formulations were similar with the exception that local, transient and usually mild nasal/pharyngeal adverse events were more common in the two intranasal formulations than in the Diastat® formulation. Following Diastat® administration, but not after the intranasal formulations, heart rate decreased about 9 to 10 bpm and systolic and diastolic blood pressure each decreased about 22-26 mmHg. These changes were also present in the 5 subjects who exhibited very poor or poor diazepam bioavailability following rectal administration, suggesting that the decreases in heart rate and blood pressure may have resulted from the rectal mode of administration, rather than a systemic pharmacologic effect of diazepam.

### Example 2

The objective of this study was to characterize the Bidose Diazepam Nasal Spray via droplet size distribution as measured by laser diffraction using a Malvern Spraytec.

DNZS Formula 1 (see, Table 1) and DNZS Formula 2 (see, Table 2) were filled in the Pfeiffer Bidose pumps fitted with two different types of vial holders. All spray pumps were automatically actuated using a SprayVIEW NSx Automated Actual Station. Droplet size distributions were measured using a Malvern Spraytec. The actuation parameters for Bidose Nasal Spray Pump were provided by the device manufacturer. The software parameters for SprayVIEW NSP were derived from our previous experience with similar types of devises.

The Malvern Spraytec operates based on laser diffraction principle and is a commonly used technique to characterize droplet size distributions from nasal sprays. The droplet size distribution is characterized by the following metrics: volume distribution (Dv10, Dv50, Dv90), Span and percentage (%) less than 10 μm per the FDA Guidance for Industry: Nasal Spray and Inhalation Solution, Suspension, and Spray Drug Products-Chemistry, Manufacturing and Controls Documentation, July 2002 and FDA Draft Guidance for Industry: Bioavailability and Bioequivalence Studies for Nasal Aerosols and Nasal Sprays for Local Action, April 2003.

### Definitions

Actuation: The process of discharging a nasal spray.
Spray Weight: The weight of formulation emitted from a nasal spray unit by a single actuation (Initial Unit Weight−Final Unit Weight). The target spray weight for the Bi-Dose Diazepam Nasal Spray is approximately 100 mg.
Dv50: The volume median diameter or Dv50 value indicates that 50% of the distribution is contained in droplets that are smaller than this value while the other half is contained in droplets that are larger than this value. Similarly the Dv10 and Dv90 values indicate that 10% and 90%, respectively, of the distribution is contained in droplets that are smaller than these values.
Span: The span is measured during laser diffraction testing. It quantifies the spread of the droplet size distribution and is calculated by the following equation: Dv90−Dv10/Dv50.
Percentage (%) less than 10 μm: When measured by laser diffraction, the percent less than 10 μm relates to the percent of droplet size distribution that is 10 microns in diameter or smaller.

### Test Execution

The Diazepam bulk formulations were stored at room temperature and the Diazepam Nasal Spray (filled units) was stored upright at room temperature. The spray weights were recorded on spray weight spreadsheets designated to this project. All test data and observations were recorded in the assigned laboratory notebook.

### Preparation/Assembly of the Diazepam Formulations

### Vial Assembly Process

The diazepam formulations did not require shaking. Using an Eppendorf pipette, 230 μl of each formulation (DZNS Formula 1 or DZNS Formula 2) were pipetted into the each vial. Care was taken not to wet the sides while filling. The filled vial was inserted into the metal vial holder. The rubber stopper was inserted into the rubber stopper holder until the upper surfaces of the holder and stopper were even. The rubber stopper holder was placed vertically onto the metal vial holder. The assembly shell was placed vertically onto the rubber stopper holder. The assembly shell was then fully depressed to insert the rubber stopper into the vial. The assembly shell and the rubber stopper holder were removed. The vial was removed from the metal vial holder by turning the metal vial holder upside down.

### Bidose Device Assembly Process

A plastic vial holder was placed vertically under the filled vial (now called a vial holder assembly). The vial holder assembly was placed into the final assembly aid. The Bidose pre-assembly was placed onto the vial holder. The pre-assembly was fully pushed down on to the assembly aid so that the lower edge of the adapter touched the aid.

### Method for Determining Droplet Size Distribution Bidose Diazepam Nasal Spray

The actuation and software parameters described in Table 6 were used for droplet size distribution using the SprayVIEW NSx-MS and Malvern Spraytec.

TABLE 6
Actuation Parameters for the SprayVIEW NSx Actuation Station
and Software Parameters for the Malvern Spraytec

| | |
|---|---|
| Instrument Setting | Input Parameter |

SprayVIEW NSx Actuation Station

| Profile | Symmetric | |
|---|---|---|
| Spray #1 Stroke Length | 16.0 | mm |
| Spray #2 Stroke Length | 10.0 | mm |
| Velocity | 50 | mm/sec |
| Acceleration | 3000 | mm/sec² |
| Initial Delay | 30 | msec |
| Final Delay | 0 | msec |
| Hold Time | 100 | ms |

Malvern Spraytec

| | | |
|---|---|---|
| Test Duration | 300 | ms |
| Data Acquisition Rate | 1000 | Hz |

| | |
|---|---|
| Acquisition Duty Cycle | 50% |
| Experimental Trigger | Level |
| Level Trigger | 20% |
| Trigger Source | None |
| Transmission Filter | 95% |
| Stable Phase Selection | Manual |

The Pfeiffer devices were filled and assembled. A total of 12 units were selected. The initial unit weights were recorded. The droplet sizes of a two actuations per unit were measured. The tip was wiped with a Kimwipe and each unit was weighed after each spray to calculate each spray weight. The stable phase was manually selected by the analyst from the acquired histogram for each actuation to analyze the droplet size distribution (DSD). From the Malvern Spraytec Toolbar; the analyst selected View and highlighted Relative Timing. The Malvern Spraytec Process Control Variable File (.pcl) and Data File (.dat) were saved. The Malvern Spraytec Cover Page, PSD and PCV table were printed. Data was recorded in the Spray Weight Worksheet, laboratory notebook and Malvern Spraytec. The Dv10, Dv50, Dv90, Span, %<10 μm and Spray Weight were reported.

### Results and Discussion

The objective of this study was to characterize two formulations of Bidose Diazepam Nasal Spray supplied in Pfeiffer Bidose pumps fitted with two different types of vial holders. DZNS Formula 2 is a high viscosity formulation and DZNS Formula 1 is a low viscosity formulation. Both DZNS Formula 1 and DZNS Formula 2 were tested with a standard as well as a modified vial holder. This modified vial holder was designed to improve the plume profile of these formulations by increasing the pressure point of Bidose at the time of actuation, as per the device manufacturer (Pfeiffer).
In-vitro spray characterization of the two formulations was based on spray pattern analysis as measured by a Malvern Spraytec. A total of 24 actuations were tested by one analyst (3 Devices×2 Formulations×2 types of Vial Holders×2 Actuations).
Refer to Tables 7 and 8 below for the droplet size averages generated from modified and standard vial holders. The data comparison can be found in Table 9,

TABLE 7
Overall Droplet Size Averages from Modified Vial Holders

| | | | | | | Spray |
|---|---|---|---|---|---|---|
| | Dv10 | Dv50 | Dv90 | | | Weight |
| | (μm) | (μm) | (μm) | Span | % < 10 μm | (mg) |
| | Formula 2 (Modified Vial Holders) | | | | | |
| Overall | 105.11 | 241.35 | 389.95 | 1.19 | 0.08 | 97.8 |
| Average | | | | | | |
| Overall SD | 15.15 | 23.74 | 18.85 | 0.11 | 0.03 | 5.4 |
| % CV | 14.4 | 9.8 | 4.8 | 9.2 | 42.7 | 5.5 |
| | Formula 1 (Modified Vial Holders) | | | | | |
| Overall | 20.38 | 44.29 | 99.86 | 1.79 | 2.04 | 98.0 |
| Average | | | | | | |
| Overall SD | 0.78 | 1.64 | 5.39 | 0.05 | 0.19 | 4.4 |
| % CV | 3.8 | 3.7 | 5.4 | 2.8 | 9.5 | 4.5 |
| | Formula 1 + Formula 2 (Modified Vial Holders) | | | | | |
| Overall | 62.74 | 142.82 | 244.90 | 1.49 | 1.06 | 97.9 |
| Average | | | | | | |
| Overall SD | 45.42 | 104.15 | 152.07 | 0.33 | 1.03 | 4.7 |
| % CV | 72.4 | 72.9 | 62.1 | 21.8 | 97.2 | 4.8 |

TABLE 8
Overall Droplet Size Averages from Standard Vial Holders

| | | | | | | Spray |
|---|---|---|---|---|---|---|
| | Dv10 | Dv50 | Dv90 | | | Weight |
| | (μm) | (μm) | (μm) | Span | % < 10 μm | (mg) |
| | DZNS Formula 2 (Standard Vial Holders) | | | | | |
| Overall | 136.22 | 276.62 | 410.86 | 1.00 | 0.08 | 95.3 |
| Average | | | | | | |
| Overall SD | 11.69 | 14.29 | 7.81 | 0.07 | 0.03 | 9.3 |
| % CV | 8.6 | 5.2 | 1.9 | 7.0 | 35.3 | 9.8 |
| | DZNS Formula 1 (Standard Vial Holders) | | | | | |
| Overall | 20.44 | 45.62 | 106.01 | 1.87 | 1.99 | 98.6 |
| Average | | | | | | |
| Overall SD | 1.27 | 3.60 | 12.76 | 0.11 | 0.37 | 14.2 |
| % CV | 6.2 | 7.9 | 12.0 | 5.6 | 18.8 | 14.5 |
| | DZNS Formula 1 + DZNS Formula 2 | | | | | |
| | (Standard Vial Holders) | | | | | |
| Overall | 78.33 | 161.12 | 258.43 | 1.43 | 1.04 | 96.9 |
| Average | | | | | | |
| Overall SD | 60.98 | 121.05 | 159.52 | 0.46 | 1.03 | 11.6 |
| % CV | 77.9 | 75.1 | 61.7 | 32.4 | 99.1 | 12.0 |

TABLE 9
Comparison between DZNS Formula 1 and DZNS Formula 2 when tested with
modified vial holder and standard vial holder

| | Holder | | | | | | Weight |
|---|---|---|---|---|---|---|---|
| Formulation | Type | Dv10 (μm) | Dv50 (μm) | Dv90 (μm) | Span | % < 10 μm | (mg) |
| DZNS | Modified | 105.11 ± 15.15 | 241.35 ± 23.74 | 389.95 ± 18.85 | 1.19 ± 0.11 | 0.08 ± 0.03 | 97.8 ± 5.4 |
| Formula 2 | Standard | 136.22 ± 11.69 | 276.62 ± 14.29 | 410.86 ± 7.81 | 1.00 ± 0.07 | 0.08 ± 0.03 | 95.3 ± 9.3 |
| DZNS | Modified | 20.38 ± 0.78 | 44.29 ± 1.64 | 99.86 ± 5.39 | 1.79 ± 0.05 | 2.04 ± 0.19 | 98.0 ± 4.4 |
| Formula 1 | standard | 20.44 ± 1.27 | 45.62 ± 3.60 | 106.01 ± 12.76 | 1.87 ± 0.11 | 1.99 ± 0.37 | 98.6 ± 14.2 |

As shown in Table 9, the droplet size data of DZNS Formula 1 and DZNS Formula 2 were observed to be considerably different. The Dv10, Dv50, and Dv90 values obtained from DZNS Formula 2 were higher than those obtained from DZNS Formula 1. Without being bound to a particular theory, this could be due to the fact that the high viscosity DZNS Formula 2 resulted in a stream-like spray with large droplet particles (including sputter) and the low viscosity formulation DZNS Formula 1 resulted in a better developed plume resulting in much smaller droplet particles. Subsequently, the DZNS Formula 1 resulted in a better span (more spread out of the plume) and higher %<10 μm compared to DZNS Formula 2 (more % droplet size distribution that is 10 microns in diameter or smaller). This data indicates that there is a significant effect of viscosity on the droplet size distribution of these formulations.

As per information obtained from the device manufacturer, the modified vial holder was designed to increase the pressure point of the Bidose Device, thereby resulting in a less stream-like spray from DZNS Formula 2. However, the overall droplet size distribution data from Modified vial holder was comparable to that from the Standard vial holder.

### Summary and Conclusions

All sprays actuated met the acceptance limits as defined by single actuation content of 85 to 115% of the target spray weight (100 mg) which therefore indicates that a fully developed spray was analyzed.

### Example 3

The objective of this study was to characterize the Bidose Diazepam Nasal Spray via plume geometry analysis as measured by a SprayVIEW NSP.

DNZS Formula 1 (see, Table 1) and DNZS Formula 2 (see, Table 2) were filled in the Pfeiffer Bidose pumps fitted with two different types of vial holders. All spray pumps were automatically actuated using a SprayVIEW NSx Automated Actual Station. Plume geometries were measured using a SprayVIEW NSP. The actuation parameters for Bidose Nasal Spray Pump were provided by the device manufacturer. The software parameters for SprayVIEW NSP were derived from our previous experience with similar types of devices.

Plume geometry is an in vitro test used to characterize pump performance. This test is performed from the analysis of a two-dimensional image of the emitted plum. Plume geometry analysis will be performed using SprayVIEW NSP, which is a non-impaction laser sheet-based instrument. The plume geometry is characterized by the following metric: spray angle and plume width per FDA Guidance for Industry: Nasal Spray and Inhalation Solution, Suspension, and Spray Drug Products—Chemistry, Manufacturing and Controls Documentation, July 2002 and FDA Draft Guidance for Industry: Bioavailability and Bioequivalence Studies for Nasal Aerosols and Nasal Sprays for Local Action, April 2003.

### Definitions

Actuation: The process of discharging a nasal spray.
Spray Weight: The weight of formulation emitted from a nasal spray unit by a single actuation (Initial Unit Weight−Final Unit Weight). The target spray weight for the Bi-Dose Diazepam Nasal Spray is approximately 100 mg.
Spray Angle: The angle of the emitted plume measured from the vertex of the spray cone and spray nozzle.
Plume Width: The width of the plume at a given distance from the spray nozzle. For this study, plume width will be measured at 3 cm plume width distance from the spray nozzle.

### Test Execution

The Diazepam bulk formulations were stored at room temperature and the Diazepam Nasal Spray units (filled) were stored upright at room temperature. The spray weights were recorded on spray weight spreadsheets designated to this project. All test data and observations were recorded in the assigned laboratory notebook.

### Preparation/Assembly of the Diazepam Formulations

### Vial Assembly Process

The diazepam formulations did not require shaking. Using an Eppendorf pipette, 230 μl of each formulation (DZNS Formula 1 or DZNS Formula 2) were pipetted into the each vial. Care was taken not to wet the sides while filling. The filled vial was inserted into the metal vial holder. The rubber stopper was inserted into the rubber stopper holder until the upper surfaces of the holder and stopper were even. The rubber stopper holder was placed vertically onto the metal vial holder. The assembly shell was placed vertically onto the rubber stopper holder. The assembly shell was then fully depressed to insert the rubber stopper into the vial. The assembly shell and the rubber stopper holder were removed. The vial was removed from the metal vial holder by turning the metal vial holder upside down.

### Bidose Device Assembly Process

A plastic vial holder was placed vertically under the filled vial (now called a vial holder assembly). The vial holder assembly was placed into the final assembly aid. The Bidose pre-assembly was placed onto the vial holder. The pre-assembly was fully pushed down on to the assembly aid so that the lower edge of the adapter touched the aid.

### Method for Determining Plume Geometry Biodose Diazepam Nasal Spray

The actuation and software parameters described in Table 10 were used for plume geometry using the SprayVIEW NSx and SprayVIEW NSP.

TABLE 10
Actuation Parameters for the SprayVIEW NSP and Software
Parameters for the SprayVIEW NSx Actuation Station

| | |
|---|---|
| Instrument Setting | Input Parameter |

SprayVIEW NSx Actuation Station

| Profile | Symmetric | |
|---|---|---|
| Spray #1 Stroke Length | 16.0 | mm |
| Spray #2 Stroke Length | 10.0 | mm |
| Velocity | 50 | mm/sec |
| Acceleration | 3000 | mm/sec² |
| Initial Delay | 30 | msec |
| Final Delay | 0 | msec |
| Hold Time | 100 | ms |

SprayVIEW NSP

| Plume Width Distance | 3 cm (30 mm) | |
|---|---|---|
| Frame Rate | 125 | Hz |
| Number of Images to Acquire | 500 | |
| Lens Aperture | 2.0 | |
| Camera Position Vertical (from the top) | 9.6 | cm |
| Camera Position Horizontal (from the left) | 23.0 | cm |
| Laser Position Vertical (from the top) | 14.9 cm (Set the calibration | |
| | grid to touch the tip of | |
| | the actuator) | |
| Laser Position Horizontal (from the left) | 10.0 | cm |

| | |
|---|---|
| Plume Orientation (degrees) | 0 |
| Time Delay (Frame) | Select from plateau |
| | region and record |
| Arms 1 & 2 (%) | Set visually and report |
| Palette | Gradient |

The Pfeiffer devices were filled and assembled. A total of 12 units were selected. The initial unit weights were recorded. The plume geometries of two actuations per unit were measured. The tip was wiped with a Kimwipe and each unit was weighed after each spray to calculate each spray weight. The SprayVIEW Plume Geometry Reports were printed. Data was recorded in the Spray Weight Worksheet, laboratory notebook and SprayVIEW NSP. The Spray Angle, Plume Width and Spray Weights were reported.

### Results and Discussion

The objective of this study was to characterize two formulations of Bidose Diazepam Nasal Spray supplied in Pfeiffer Bidose pumps fitted with two different types of vial holders. DZNS Formula 2 is a high viscosity formulation and DZNS Formula 1 is a low viscosity formulation as per. Both DZNS Formula 1 and DZNS Formula 2 were tested with a standard as well as a modified vial holder. This modified vial holder was designed to improve the plume profile of these formulations by increasing the pressure point of Bidose at the time of actuation, as per the device manufacturer (Pfeiffer).

In-vitro spray characterization of the two formulations was based on plume geometry analysis as measured by a SprayVIEW NSP. A total of 24 actuations were tested by one analyst (3 Devices×2 Formulations×2 types of Vial Holders×2 Actuations).

Refer to Tables 11 and 12 below for the plume geometry averages generated from modified and standard vial holders. The data comparison can be found in Table 13.

TABLE 11
Overall Plume Geometry Averages from Modified Vial Holders
Data Summary

| | Spray | Plume | Spray |
|---|---|---|---|
| | Angle (°) | Width (mm) | Weight (mg) |
| | DZNS Formula 2 (Modified Vial Holders) | | |
| Overall Average | 292 | 15.7 | 98.4 |
| Overall SD | 8.9 | 5.1 | 9.4 |
| % CV | 30.6 | 32.6 | 9.5 |
| | DZNS Formula 1 (Modified Vial Holders) | | |
| Overall Average | 74.8 | 46.1 | 98.3 |
| Overall SD | 1.7 | 1.4 | 5.9 |
| % CV | 2.3 | 3.1 | 6.0 |
| | DZNS Formula 1 + DZNS Formula 2 | | |
| | (Modified Vial Holders) | | |
| Overall Average | 52.0 | 30.9 | 98.3 |
| Overall SD | 24.6 | 16.2 | 7.5 |
| % CV | 47.3 | 52.6 | 7.6 |

TABLE 12
Overall Plume Geometry Averages from Standard Vial Holders
Data Summary

| | Spray | Plume | Spray |
|---|---|---|---|
| | Angle (°) | Width (mm) | Weight (mg) |
| | DZNS Formula 2 (Standard Vial Holders) | | |
| Overall Average | 32.6 | 17.7 | 95.7 |
| Overall SD | 5.2 | 3.0 | 11.8 |
| % CV | 16.1 | 17.1 | 12.3 |
| | DZNS Formula 1 (Standard Vial Holders) | | |
| Overall Average | 70.2 | 42.2 | 98.4 |
| Overall SD | 1.7 | 1.4 | 12.5 |
| % CV | 2.5 | 3.2 | 12.8 |
| | DZNS Formula 1 + DZNS Formula 2 | | |
| | (Standard Vial Holders) | | |
| Overall Average | 51.4 | 29.9 | 97.0 |
| Overall SD | 20.0 | 13.0 | 11.7 |
| % CV | 38.8 | 43.5 | 12.1 |

TABLE 13
Comparison between DZNS Formula 1 and DZNS Formula 2 when
tested with modified vial holder & standard vial holder

| | Vial Holder | Spray | Plume | Spray |
|---|---|---|---|---|
| Formulation | Type | Angle (°) | Width (mm) | Weight (mg) |
| DZNS Formula 2 | Modified | 29.2 ± 8.9 | 15.7 ± 5.1 | 98.4 ± 9.4 |
| (High Viscosity) | Standard | 32.6 ± 5.2 | 17.7 ± 3.0 | 95.7 ± 11.8 |
| DZNS Formula 1 | Modified | 74.8 ± 1.7 | 46.1 ± 1.4 | 98.3 ± 5.9 |
| (Low Viscosity) | Standard | 70.2 ± 1.7 | 42.2 ± 1.4 | 98.4 ± 12.5 |

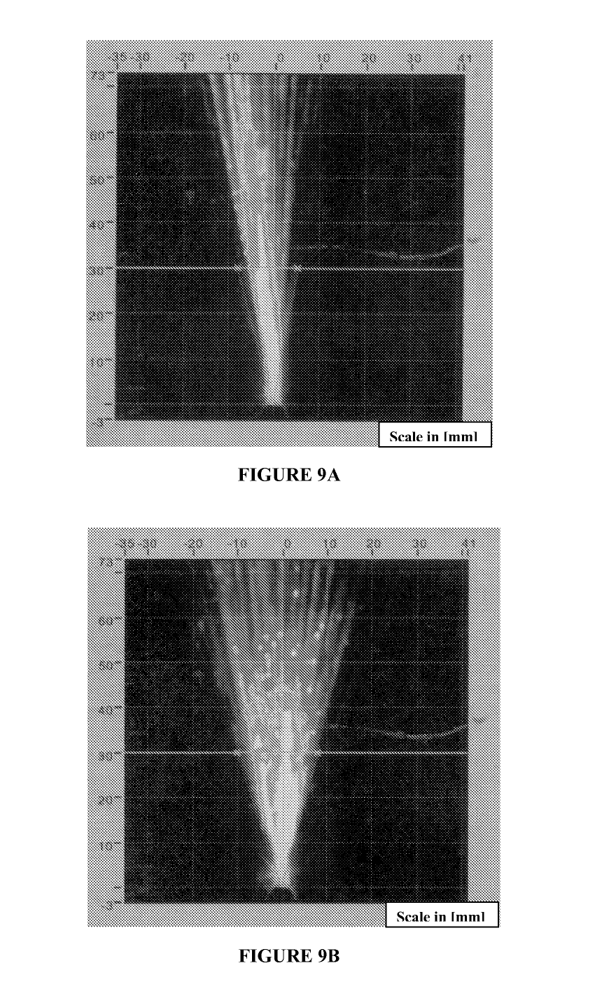

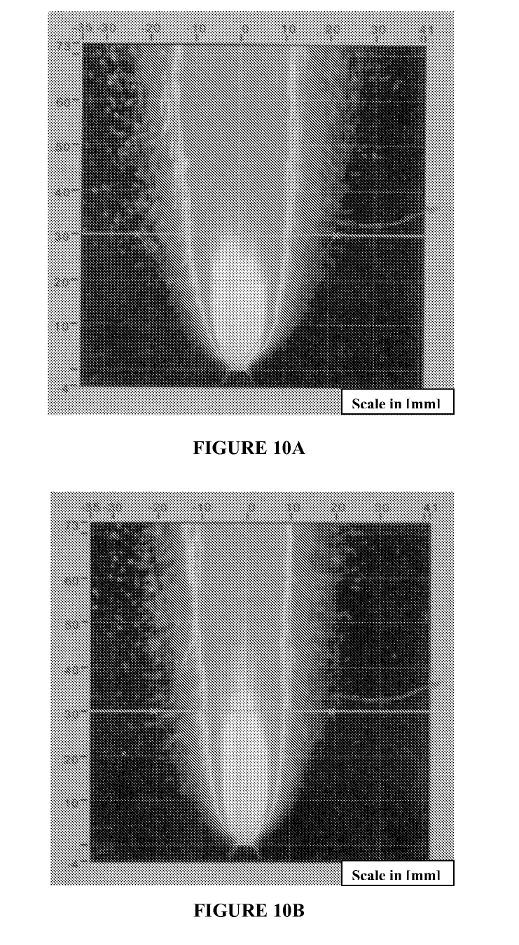

As shown in Table 13 and FIGS. 9-10, the plume geometry data of DZNS Formula 1 and DZNS Formula 2 were observed to be considerably different. The spray angle and plume width values obtained from DZNS Formula 2 were lower than those obtained from DZNS Formula 1. Without being bound to a particular theory, this could be due to the fact that the high viscosity formulation DZNS Formula 2 resulted in a stream-like spray (narrow plume) and the low viscosity formulation DZNS Formula 1 resulted in a better developed plume resulting in a bigger plume size and broader angle.

This data indicates that there is a significant effect of viscosity on the plume geometry characteristics of these formulations when dispensed using the Pfeiffer Bidose device.

As per information obtained from the device manufacturer, the modified vial holder was designed to increase the pressure point of the Bidose Device, thereby resulting in a less stream-like spray from DZNS Formula 2. However, the overall spray pattern data from the modified vial holder was comparable to that from the standard vial holder.

### Summary and Conclusions

All sprays actuated met the acceptance limits as defined by single actuation content of 85 to 115% of the target spray weight (100 mg) which therefore indicates that a fully developed spray was analyzed.

### Example 4

The objective of this study was to characterize the Bidose Diazepam Nasal Spray via spray pattern analysis as measured by a SprayVIEW NSP.

DNZS Formula 1 (see, Table 1) and DNZS Formula 2 (see, Table 2) were filled in the Pfeiffer Bidose pumps fitted with two different types of vial holders. All spray pumps were automatically actuated using a SprayVIEW NSx Automated Actual Station. Spray patterns were measured using a SprayVIEW NSP. The actuation parameters for Bidose Nasal Spray Pump were provided by the device manufacturer. The software parameters for SprayVIEW NSP were derived from our previous experience with similar types of devices.

Spray pattern is an in vitro test used to characterize pump performance. This test is performed from the analysis of a two-dimensional image of the emitted plum. Spray pattern will be performed using SprayVIEW NSP, which is a non-impaction laser sheet-based instrument. The spray pattern is characterized by the following metrics: Dmax, Dmin, and Ovality Ratio per the FDA Guidance for Industry: Nasal Spray and Inhalation Solution, Suspension, and Spray Drug Products—Chemistry, Manufacturing and Controls Documentation, July 2002 and FDA Draft Guidance for Industry: Bioavailability and Bioequivalence Studies for Nasal Aerosols and Nasal Sprays for Local Action, April 2003.

### Definitions

Actuation: The process of discharging a nasal spray.
Spray Weight: The weight of formulation emitted from a nasal spray unit by a single actuation (Initial Unit Weight−Final Unit Weight). The target spray weight for the Bi-Dose Diazepam Nasal Spray is approximately 100 mg.
Dmax: The longest diameter measured on the resulting spray pattern image. The Dmax must pass through the center (weighted for image intensity) of the spray pattern image.
Dmin: The shortest diameter measured on the resulting spray pattern image. The Dmin must pass through the center (weighted for image intensity) of the spray pattern image.
Ovality Ratio: The ratio of Dmax to Dmin. This ratio provides a quantitative value for the overall shape of the spray.
Percent Area: The ratio of the spray pattern area to the entire image area (%).

### Test Execution

The Diazepam bulk formulations were stored at room temperature and the Diazepam Nasal Spray units (filled) were stored upright at room temperature. The spray weights were recorded on spray weight spreadsheets designated to this project. All test data and observations were recorded in the assigned laboratory notebook.

### Preparation/Assembly of the Diazepam Formulations

### Vial Assembly Process

The diazepam formulations did not require shaking. Using an Eppendorf pipette, 230 μl of each formulation (DZNS Formula 1 or DZNS Formula 2) were pipetted into the each vial. Care was taken not to wet the sides while filling. The filled vial was inserted into the metal vial holder. The rubber stopper was inserted into the rubber stopper holder until the upper surfaces of the holder and stopper were even. The rubber stopper holder was placed vertically onto the metal vial holder. The assembly shell was placed vertically onto the rubber stopper holder. The assembly shell was then fully depressed to insert the rubber stopper into the vial. The assembly shell and the rubber stopper holder were removed. The vial was removed from the metal vial holder by turning the metal vial holder upside down.

### Bidose Device Assembly Process

A plastic vial holder was placed vertically under the filled vial (now called a vial holder assembly). The vial holder assembly was placed into the final assembly aid. The Bidose pre-assembly was placed onto the vial holder. The pre-assembly was fully pushed down on to the assembly aid so that the lower edge of the adapter touched the aid.

### Method for Determining Spray Pattern Biodose Diazepam Nasal Spray

The actuation and software parameters described in Table 14 were used for spray pattern using the SprayVIEW NSx and SprayVIEW NSP.

TABLE 14
Acuation Parameters for the SprayVIEW NSP and Software
Parameters for the SprayVIEW NSx Station

| | |
|---|---|
| Instrument Setting | Input Parameter |

SprayVIEW NSx Actuation Station

| Profile | Symmetric | |
|---|---|---|
| Spray #1 Stroke Length | 16.0 | mm |
| Spray #2 Stroke Length | 10.0 | mm |
| Velocity | 50 | mm/sec |
| Acceleration | 3000 | mm/sec² |
| Initial Delay | 30 | msec |
| Final Delay | 0 | msec |
| Hold Time | 100 | ms |

SprayVIEW NSP

| Distance to the Laser Beam | 3 cm (30 mm) | |
|---|---|---|
| (Orifice to Tip Distance) | | |
| Frame Rate | 125 | Hz |
| Number of Images to Acquire | 500 | |
| Lens Aperture | 2.0 | |
| Camera Position Vertical | 33 | cm |
| (top of truck) | | |
| Camera Position Horizontal | 7 | cm |
| (right of truck) | | |
| Laser Position Horizontal | 8.6 | cm |
| (left of truck) | | |

| | |
|---|---|
| Analysis Method | Automatic |
| Palette | Gradient |

The Pfeiffer devices were filled and assembled. A total of 12 units were selected. The initial unit weights were recorded. The spray patterns of two actuations per unit were measured. The tip was wiped with a Kimwipe and each unit was weighed after each spray to calculate each spray weight. The SprayVIEW Spray Pattern Reports were printed. Data was recorded in the Spray Weight Worksheet, laboratory notebook and SprayVIEW. The Dmin, Dmax, Ovality Ratio, Percent Area and Spray Weights were reported,

### Results and Discussion

The objective of this study was to characterize two formulations of Bidose Diazepam Nasal Spray supplied in Pfeiffer Bidose pumps fitted with two different types of vial holders. DZNS Formula 2 is a high viscosity formulation and DZNS Formula 1 is a low viscosity formulation as per. Both DZNS Formula 1 and DZNS Formula 2 were tested with a standard as well as a modified vial holder. This modified vial holder was designed to improve the plume profile of these formulations by increasing the pressure point of Bidose at the time of actuation, as per the device manufacturer (Pfeiffer).

In-vitro spray characterization of the two formulations was based on spray pattern analysis as measured by a SprayVIEW NSP. A total of 24 actuations were tested by one analyst (3 Devices×2 Formulations×2 types of Vial Holders×2 Actuations).

Refer to Tables 15 and 16 below for the spray pattern averages generated from modified and standard vial holders. The data comparison can be found in Table 17.

TABLE 15
Overall Spray Pattern Averages from Modified Vial Holders
Data Summary

| | Dmax | Dmin | Ovality | % | Spray |
|---|---|---|---|---|---|
| | (mm) | (mm) | Ratio | Area | Weight (mg) |
| | DZNS Formula 2 (Modified Vial Holders) | | | | |
| Overall Average | 9.2 | 4.3 | 2.375 | 0.8 | 97.8 |
| Overall SD | 0.8 | 1.6 | 0.869 | 0.4 | 3.6 |
| % CV | 9.2 | 38.1 | 36.6 | 48.3 | 3.7 |
| | DZNS Formula 1 (Modified Vial Holders) | | | | |
| Overall Average | 21.8 | 16.7 | 1.301 | 7.1 | 98.5 |
| Overall SD | 1.6 | 0.8 | 0.112 | 0.5 | 6.1 |
| % CV | 7.1 | 4.6 | 8.6 | 7.5 | 6.2 |
| | DZNS Formula 1 + DZNS Formula 2 | | | | |
| | (Modified Vial Holders) | | | | |
| Overall Average | 15.5 | 10.5 | 1.838 | 3.9 | 98.1 |
| Overall SD | 6.7 | 6.6 | 0.815 | 3.3 | 4.8 |
| % CV | 43.3 | 62.8 | 44.3 | 85.1 | 4.9 |

TABLE 16
Overall Spray Pattern Averages from Standard Vial Holders
Data Summary

| | Dmax | Dmin | Ovality | % | Spray |
|---|---|---|---|---|---|
| | (mm) | (mm) | Ratio | Area | Weight (mg) |
| | DZNS Formula 2 (Standard Vial Holders) | | | | |
| Overall Average | 12.2 | 6.0 | 2.129 | 1.5 | 95.4 |
| Overall SD | 1.8 | 1.2 | 0.526 | 0.3 | 10.6 |
| % CV | 14.9 | 19.8 | 24.7 | 22.3 | 11.1 |
| | DZNS Formula 1 (Standard Vial Holders) | | | | |
| Overall Average | 22.5 | 17.4 | 1.298 | 7.5 | 97.0 |
| Overall SD | 1.7 | 0.8 | 0.108 | 0.6 | 12.7 |
| % CV | 7.4 | 4.4 | 8.3 | 8.7 | 13.1 |
| | DZNS Formula 1 + DZNS Formula 2 | | | | |
| | (Standard V0.6ial Holders) | | | | |
| Overall Average | 17.4 | 11.7 | 1.713 | 4.5 | 96.2 |
| Overall SD | 5.6 | 6.0 | 0.565 | 3.2 | 11.2 |
| % CV | 32.4 | 51.8 | 33.0 | 70.4 | 11.6 |

TABLE 17
Comparison between DZNS Formula 1 and DZNS Formula 2 when tested with
modified vial holders & standard vial holders.

| | Vial | | | | | |
|---|---|---|---|---|---|---|
| | Holder | Dmax | Dmin | Ovality | | Spray Weight |
| Formulation | Type | (mm) | (mm) | Ratio | % Area | (mg) |
| DZNS Formula 2 | Modified | 9.2 ± 0.8 | 4.3 ± 1.6 | 2.375 ± 0.869 | 0.8 ± 0.4 | 97.8 ± 3.6 |
| | Standard | 12.2 ± 1.8 | 6.0 ± 1.2 | 2.129 ± 0.526 | 1.5 ± 0.3 | 95.4 ± 10.6 |
| DZNS Formula 1 | Modified | 21.8 ± 1.6 | 16.7 ± 0.8 | 1.301 ± 0.112 | 7.1 ± 0.5 | 98.5 ± 6.1 |
| | standard | 22.5 ± 1.7 | 17.4 ± 0.8 | 1.298 ± 0.108 | 7.5 ± 0.6 | 97.0 ± 12.7 |

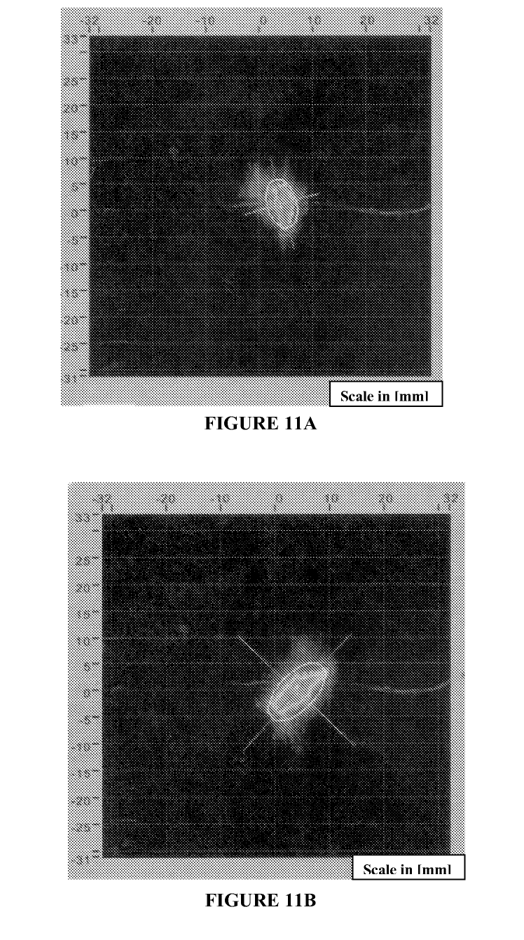

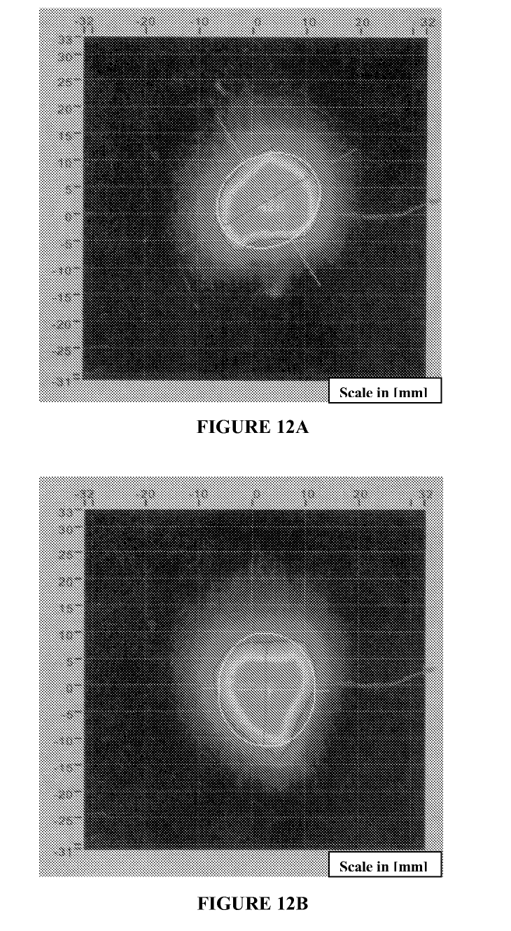

As shown in Table 17 and FIGS. 11-12, the spray pattern data of DZNS Formula 1 and DZNS Formula 2 were observed to be considerably different. The Dmax, Dmin, and % Area values obtained from DZNS Formula 1 were higher than those obtained from DZNS Formula 2. This could be due to the fact that the high viscosity of formulation DZNS Formula 2 resulted in a stream-like spray with low Dmax, Dmin, and % Area values and the low viscosity formulation resulted in a better developed plume resulting in larger spray patterns. Subsequently, DZNS Formula 1 resulted in a better Ovality Ratio compared to DZNS Formula 2. (An Ovality ratio of 1 is regarded as a perfectly circular pattern).

This data indicates that there is a significant effect of viscosity on the spray pattern characteristics of these formulations when dispensed using the Pfeiffer Bidose device.

As per information obtained from the device manufacturer, the modified vial holder was designed to increase the pressure point of the Bidose Device, thereby resulting in a less stream-like spray from DZNS Formula 2. However, the overall spray pattern data from Modified vial holder was comparable to that from the Standard vial holder.

### Summary and Conclusions

All sprays actuated met the acceptance limits as defined by single actuation content of 85 to 115% of the target spray weight (100 mg) which therefore indicates that a fully developed spray was analyzed.

### Example 5

The following formulations were prepared and/or contemplated with various concentrations of diazepam and other components. In some embodiments, the formulations are to allow for proper per weight dosing in patients per the label. In other embodiments, the formulations are to improve the solubility and/or bioavailability of diazepam.

### Formulation 1

| Ingredient (Trade Name) | % wt/wt |
|---|---|
| Diazepam | 2.50 |
| Diethylene glycol monoethyl ether, NF (Transcutol ® HP) | 48.20 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

### Formulation 2

| Ingredient (Trade Name) | % wt/wt |
|---|---|
| Diazepam | 3.75 |
| Diethylene glycol monoethyl ether, NF (Transcutol ® HP) | 46.95 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

### Formulation 3

| Ingredient (Trade Name) | % wt/wt |
|---|---|
| Diazepam | 5.00 |
| Diethylene glycol monoethyl ether, NF (Transcutol ® HP) | 45.70 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

### Formulation 4

| Ingredient (Trade Name) | % wt/wt |
|---|---|
| Diazepam | 6.25 |
| Diethylene glycol monoethyl ether, NF (Transcutol ® HP) | 44.45 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

### Formulation 5

| Ingredient (Trade Name) | % wt/wt |
|---|---|
| Diazepam | 7.50 |
| Diethylene glycol monoethyl ether, NF (Transcutol ® HP) | 43.20 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

### Formulation 6

| Ingredient (Trade Name) | % wt/wt |
|---|---|
| Diazepam | 8.75 |
| Diethylene glycol monoethyl ether, NF (Transcutol ® HP) | 41.95 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

### Formulation 7

| Ingredient (Trade Name) | % wt/wt |
|---|---|
| Diazepam | 10.00 |
| Diethylene glycol monoethyl ether, NF (Transcutol ® HP) | 40.70 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

### Formulation 8

| Ingredient (Trade Name) | % wt/wt |
|---|---|
| Diazepam | 2.50 |
| Diethylene glycol monoethyl ether, NF (Transcutol ® HP) | 48.10 |
| Caprylocaproylpolyoxylglyceride, NF (Labrasol ®) | 30.30 |
| Sorbitan monolaurate, NF (SPAN ® 20) | 10.80 |
| Isopropyl palmitate, NF | 7.30 |
| Purified Water, USP | 1.00 |

### Formulation 9

| Ingredient (Trade Name) | % wt/wt |
|---|---|
| Diazepam | 3.75 |
| Diethylene glycol monoethyl ether, NF (Transcutol ® HP) | 46.85 |
| Caprylocaproylpolyoxylglyceride, NF (Labrasol ®) | 30.30 |
| Sorbitan monolaurate, NF (SPAN ® 20) | 10.80 |
| Isopropyl palmitate, NF | 7.30 |
| Purified Water, USP | 1.00 |

### Formulation 10

| Ingredient (Trade Name) | % wt/wt |
|---|---|
| Diazepam | 5.00 |
| Diethylene glycol monoethyl ether, NF (Transcutol ® HP) | 45.60 |
| Caprylocaproylpolyoxylglyceride, NF (Labrasol ®) | 30.30 |
| Sorbitan monolaurate, NF (SPAN ® 20) | 10.80 |
| Isopropyl palmitate, NF | 7.30 |
| Purified Water, USP | 1.00 |

### Formulation 11

| Ingredient (Trade Name) | % wt/wt |
|---|---|
| Diazepam | 6.25 |
| Diethylene glycol monoethyl ether, NF (Transcutol ® HP) | 44.35 |
| Caprylocaproylpolyoxylglyceride, NF (Labrasol ®) | 30.30 |
| Sorbitan monolaurate, NF (SPAN ® 20) | 10.80 |
| Isopropyl palmitate, NF | 7.30 |
| Purified Water, USP | 1.00 |

### Formulation 12

| Ingredient (Trade Name) | % wt/wt |
|---|---|
| Diazepam | 7.50 |
| Diethylene glycol monoethyl ether, NF (Transcutol ® HP) | 43.10 |
| Caprylocaproylpolyoxylglyceride, NF (Labrasol ®) | 30.30 |
| Sorbitan monolaurate, NF (SPAN ® 20) | 10.80 |
| Isopropyl palmitate, NF | 7.30 |
| Purified Water, USP | 1.00 |

### Formulation 13

| Ingredient (Trade Name) | % wt/wt |
|---|---|
| Diazepam | 8.75 |
| Diethylene glycol monoethyl ether, NF (Transcutol ® HP) | 41.85 |
| Caprylocaproylpolyoxylglyceride, NF (Labrasol ®) | 30.30 |
| Sorbitan monolaurate, NF (SPAN ® 20) | 10.80 |
| Isopropyl palmitate, NF | 7.30 |
| Purified Water, USP | 1.00 |

### Formulation 14

| Ingredient (Trade Name) | % wt/wt |
|---|---|
| Diazepam | 10.00 |
| Diethylene glycol monoethyl ether, NF (Transcutol ® HP) | 40.60 |
| Caprylocaproylpolyoxylglyceride, NF (Labrasol ®) | 30.30 |
| Sorbitan monolaurate, NF (SPAN ® 20) | 10.80 |
| Isopropyl palmitate, NF | 7.30 |
| Purified Water, USP | 1.00 |

### Formulation 15

| Ingredient (Trade Name) | % wt/wt |
|---|---|
| Diazepam | 4.95 |
| Diethylene glycol monoethyl ether, NF (Transcutol ® HP) | 45.62 |
| Oleoyl polyoxylglycerides (Labrafil ®) | 7.60 |
| Sorbitan monolaurate, NF (SPAN ® 20) | 11.41 |
| Caprylocaproylpolyoxylglyceride, NF (Labrasol ®) | 30.42 |

### Formulation 16

| Ingredient (Trade Name) | % wt/wt |
|---|---|
| Diazepam | 6.63 |
| Diethylene glycol monoethyl ether, NF (Transcutol ® HP) | 44.82 |
| Oleoyl polyoxylglycerides (Labrafil ®) | 7.47 |
| Sorbitan monolaurate, NF (SPAN ® 20) | 11.20 |
| Caprylocaproylpolyoxylglyceride, NF (Labrasol ®) | 29.88 |

### Formulation 17

| Ingredient (Trade Name) | % wt/wt |
|---|---|
| Diazepam | 5.00 |
| Diethylene glycol monoethyl ether, NF (Transcutol ® HP) | 45.60 |
| Caprylocaproylpolyoxylglyceride, NF (Labrasol ®) | 30.40 |
| Sorbitan monolaurate, NF (SPAN ® 20) | 10.83 |
| Isopropyl palmitate, NF | 7.22 |
| Purified Water, USP | 0.95 |

### Example 6

The following formulations were prepared in an effort to further improve the solubility and/or concentration of diazepam in the formulation and demonstrate the difficulty of achieving a suitable concentration of diazepam for intranasal administration. The formulations were, in some embodiments, compounded sequentially with diazepam added last. In particular embodiments, diazepam wasn't added until a visually clear solution was provided. In other embodiments, diazepam was added into diethylene glycol monethyl ether and sonicated for at least ten minutes, followed by the addition of the rest of the components.

### Formulation 18

| Ingredient (Trade Name) | % wt/wt |
|---|---|
| Diazepam | 10.00 |
| Diethylene glycol monoethyl ether, NF (Transcutol ® HP) | 55.00 |
| Isopropyl palmitate, NF | 5.00 |
| Sorbitan monolaurate, NF (SPAN ® 20) | 5.55 |
| Caprylocaproylpolyoxylglyceride, NF (Labrasol ®) | 23.50 |
| Purified Water, USP | 0.95 |

HPLC Analysis for % wt/wt diazepam concentration for Formulation 18 found 8.66% diazepam in the formulation.

### Formulation 19

| Ingredient (Trade Name) | % wt/wt |
|---|---|
| Diazepam | 10.00 |
| Diethylene glycol monoethyl ether, NF (Transcutol ® HP) | 60.00 |
| Isopropyl palmitate, NF | 5.00 |
| Sorbitan monolaurate, NF (SPAN ® 20) | 5.55 |
| Caprylocaproylpolyoxylglyceride, NF (Labrasol ®) | 18.50 |
| Purified Water, USP | 0.95 |

HPLC Analysis for % wt/wt diazepam concentration for Formulation 19 found 8.70% diazepam in the formulation.

### Formulation 20

| Ingredient (Trade Name) | % wt/wt |
|---|---|
| Diazepam | 10.00 |
| Diethylene glycol monoethyl ether, NF (Transcutol ® HP) | 60.00 |
| Isopropyl palmitate, NF | 5.00 |
| Sorbitan monolaurate, NF (SPAN ® 20) | 5.55 |
| Caprylocaproylpolyoxylglyceride, NF (Labrasol ®) | 18.50 |
| Purified Water, USP | 0.95 |

HPLC Analysis for % wt/wt diazepam concentration for Formulation 20 found 8.90% diazepam in the formulation.

### Formulation 21

| Ingredient (Trade Name) | % wt/wt |
|---|---|
| Diazepam | 10.00 |
| Diethylene glycol monoethyl ether, NF (Transcutol ® HP) | 70.00 |
| Isopropyl palmitate, NF | 4.00 |
| Sorbitan monolaurate, NF (SPAN ® 20) | 4.55 |
| Caprylocaproylpolyoxylglyceride, NF (Labrasol ®) | 10.50 |
| Purified Water, USP | 0.95 |

HPLC Analysis for % wt/wt diazepam concentration for Formulation 21 found 9.68% diazepam in the formulation.

### Formulation 22

| Ingredient (Trade Name) | % wt/wt |
|---|---|
| Diazepam | 10.00 |
| Diethylene glycol monoethyl ether, NF (Transcutol ® HP) | 80.00 |
| Isopropyl palmitate, NF | 2.00 |
| Sorbitan monolaurate, NF (SPAN ® 20) | 1.55 |
| Caprylocaproylpolyoxylglyceride, NF (Labrasol ®) | 5.50 |
| Purified Water, USP | 0.95 |

HPLC Analysis for % wt/wt diazepam concentration for Formulation 22 found 9.55% diazepam in the formulation.

### API Solubility Of Diazepam in Neat Excipient

| Ingredient (Trade Name) | % wt/wt |
|---|---|
| Diethylene glycol monoethyl ether, NF (Transcutol ® HP) | 9.70 |
| Isopropyl palmitate, NF | 1.25 |
| Sorbitan monolaurate, NF (SPAN ® 20) | ND |
| Caprylocaproylpolyoxylglyceride, NF (Labrasol ®) | 6.65 |
| Purified Water, USP | 0.30 |

### Formulation Process Summary

Based on the excipients listed above, Transcutol® HP demonstrated to be the best solvent for diazepam and provided solubility of diazepam at 9.72%. Each of the above formulations was prepared by subsequently increasing the percentage of Transcutol HP in the formulation. Except for Formulation 20, each of the solvent components (from Transcutol® HP to water) was compounded sequentially and provided a visually clear solution before diazepam addition. Except for Formulation 20, diazepam was added to the solvent mixture of each formulation and mixed under high speed. This process was adapted from the GMP batch manufacturing, except no API rinse using the Transcutol® HP was necessary after the API addition. After the mixing was completed, each formulation was analyzed by HPLC to determine diazepam concentration. For Formulation 20, the same formulation as Formulation 19 was used. The process for Formulation 20 was modified to add diazepam in Transcutol® HP first plus 10 min sonication of the resulted Transcutol® HP and diazepam mixture. After sonication, each of the remaining solvents was added. The mixing was continued until the end of the preparation.

### Conclusion

It may have slight effect of sonication on increasing the diazepam concentration when comparing Formulation 19 with Formulation 20. Apparently, Transcutol® HP is the only solubility enhancer in the above formulations. Increasing its concentration increased the diazepam concentration in the formulation. Without being held to a particular theory, it is believed that the diazepam concentration in the formulation is limited by the solubility of diazepam in Transcutol® HP. The maximum diazepam concentration that was obtained in the above formulations is at the solubility limit of diazepam in Transcutol® HP, which was 9.68%.

### Example 7

An Open-Label, Three-Period, Crossover Study To Determine the Relative Bioavailability of a Single 20 mg Dose of Diazepam Intranasal Spray (DZNS) Versus a Single 20 mg Dose of Diastat® (Diazepam Rectal Gel) and to Assess Pharmacokinetic Linearity for DZNS in Healthy Volunteers

### Study Objectives:

To assess the relative bioavailability (BA) of a single 20 mg intranasal (IN) dose of DZNS versus a single 20 mg rectal dose of Diastat® AcuDial™ (diazepam rectal gel)
To assess the pharmacokinetic (PK) linearity of DZNS between 5 mg and 20 mg
To evaluate the safety and tolerability of DZNS
   Study Methodology: This was a single-center, open-label, three-period, randomized, crossover study. During each dosing period, subjects were scheduled to receive one of the following treatments in a randomized order:
Single, 5 mg intranasal dose of DZNS administered as one 2.5 mg spray (100 μl) in each nostril;
Single, 20 mg intranasal dose of DZNS administered as one 10 mg spray (100 μl) in each nostril; or
Single, 20 mg dose of Diastat administered rectally
   A total of 24 healthy volunteers, male and female, were enrolled in the study. A minimum washout period of 14 days separated dose administrations.
   Diagnosis and Criteria for Inclusion: Age 18 to 50 years, inclusive; in general good health with no clinically relevant abnormalities as determined by the medical history, physical examination, electrocardiogram (ECG), and clinical laboratory results; if female, was surgically sterile, post-menopausal, or using an acceptable method of contraception; Screening body weight of 88 to 111 kg, inclusive, or Screening body weight >111 kg and body mass index (BMI)≦31 kg/m²; negative urine drug test.

### Test Formulations:

### 5 mg Intranasal Dose Formulation

| Ingredient (Trade Name) | % wt/wt |
|---|---|
| Diazepam | 2.50 |
| Diethylene glycol monoethyl ether, NF (Transcutol ® HP) | 48.20 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

### 20 mg Intranasal Dose Formulation

| Ingredient (Trade Name) | % wt/wt |
|---|---|
| Diazepam | 10.00 |
| Diethylene glycol monoethyl ether, NF (Transcutol ® HP) | 40.70 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

Duration of Treatment: One day (single-dose) during each of three 12-day dosing periods.

### Criteria for Evaluation:

Efficacy: No efficacy evaluations were performed in this Phase I study, A summary of PK analyses is provided.
Pharmacokinetic: Blood samples were collected for determination of plasma diazepam and desmethyl-diazepam (nordiazepam) concentrations using a validated method, Blood samples were drawn prior to dose administration and at 5, 10, 15, 30, and 45 minutes, and 1, 1.5, 2, 4, 6, 9, 12, 24, 48, 96, 144, 192, and 240 hours after dose administration (19 samples during each of the three dosing periods).
Safety: Safety parameters included treatment-emergent adverse events (TEAEs), clinical laboratory evaluations, vital signs, pulse oximetry, physical examinations, 12-lead ECGs, Nasal and Pharyngeal Irritation/Inflammation Assessments, Subject Alertness Observations, and the Columbia-Suicide Severity Rating Scale (C-SSRS).

### Statistical Methods:

Three analysis populations were used: The All Randomized Population consisted of all subjects who were randomized to treatment. The Safety Population consisted of subjects who took one or more doses of study drug. The PK Population consisted of all subjects who received a treatment and had adequate concentration time data to permit estimation of noncompartmental PK parameters for comparative BA or dose proportionality assessments.
The Safety Population was used for presentations of study drug administration, Study Drug Leakage Observation, AEs, clinical laboratory parameters, vital signs, pulse oximetry, 12-lead ECGs, Nasal and Pharyngeal Irritation/Inflammation Assessment, Subject Alertness Observation, and C-SSRS. The All Randomized Population was used for all other presentations and displays, except for PK data, which was presented for the PK Population.

### Analysis of Disposition, Demographics, and Safety

Standard descriptive statistics were provided for each measure and time point as follows:
Numeric variables: the number of observed values (n), mean, standard deviation (SD), median, minimum, and maximum values.
Categorical variables: count of results available and percentage of the study population at each level of a categorical variable.
   Where appropriate, change from baseline summaries were also provided and analyzed.
   The disposition summary included the number and percentage of subjects who completed the study, dosed in each treatment group; and discontinued from the study by reason for discontinuation. Demographic and baseline characteristics (age, gender, race, ethnicity, height, weight, and BMI) were summarized by treatment group using standard descriptive statistics.
   Study drug administration and leakage observation results were listed, as were concomitant medications and protocol deviations.

### Pharmacokinetic Analysis

Plasma diazepam and nordiazepam concentrations were summarized using descriptive statistics (including N, mean, SD, coefficient of variation [CV %], median, minimum, and maximum) for each treatment. The following PK parameters were estimated by noncompartmental methods from plasma samples: maximum observed plasma concentration (Cₘₐₓ), time of maximum concentration (Tₘₐₓ), area under the plasma concentration-time curve from time 0 to 24 hours after dosing calculated using the linear-up log-down trapezoidal rule (AUC₀₋₂₄), area under the plasma concentration-time curve from time 0 to time of last measurable plasma concentration calculated using the linear-up log-down trapezoidal rule (AUCₗₐₛₜ), area under the plasma concentration-time curve from time 0 extrapolated to infinity (AUC_{inf}), the percentage of the AUC that is extrapolated beyond the last measurable concentration (AUCₑₓₜ), terminal-phase rate constant estimated by linear regression of the log concentration vs. time profile (λz), terminal-phase half life (t½), apparent volume of distribution, terminal phase (Vz/F), apparent systemic clearance (CL/F), and the metabolite to parent ratio (M/P ratio). Derived plasma PK descriptive statistics were tabulated by treatment group, and summary statistics presented for PK parameters include the arithmetic and geometric mean, CV %, SD of the arithmetic mean, median, minimum, maximum, and N.
Comparison of the PK parameters Cₘₐₓ, AUC₀₋₂₄, AUCₗₐₛₜ, and AUC_{inf} for diazepam with respect to the test and reference formulations for the relative BA component was conducted using an analysis of variance (ANOVA) model with sequence, subject within sequence, treatment, and period as the classification variables using the original data and the natural logarithm of the data. Confidence intervals (CI) (90%) were constructed for the treatment ratios (test to reference) of both parameters using the log transformed data and the two one-sided test procedure. The point estimates and the CI for the log-transformed data were presented following exponentiation to the original scale.
The dose proportionality between the 5 mg DZNS dose and the 20 mg DZNS dose was assessed by dose-normalizing the Cₘₐₓ, AUC₀₋₂₄, and AUC_{inf} results and by comparing the calculated CL/F values between the two doses.
Because of results seen in a previous DZNS study, it was planned that a subset analysis of relative BA using the two one-sided test procedure would be conducted using only subjects with good BA following Diastat administration and excluding subjects with poor BA following Diastat administration (if this was observed).

### Safety Analysis

Adverse events were summarized by treatment group in an overall summary, by system organ class and preferred term (PT), by PT, by intensity, and by relatedness to study drug. Both mean observed and mean change from pre-dose vital signs and pulse oximetry values were summarized by treatment group and time point. The number and percentage of subjects with nasal and/or pharyngeal irritation/inflammation or reported discomfort were summarized by treatment group and time point. The number and percentage of subjects with each of the four levels of alertness (alert, drowsy, sleeping but arousable, and sleeping not arousable) were summarized by treatment group and time point. Laboratory parameters, interpretations of 12-lead ECGs, abnormal physical examination results, and any positive C-SSRS findings were listed.

### Demographic and Disposition Results:

A total of 24 subjects were randomized in the study and 20 subjects completed all three study periods. Two of the 4 discontinued subjects withdrew consent and the other 2 were discontinued due to protocol non-compliance. A total of 22 subjects received 5 mg DZNS and 23 subjects received both 20 mg DZNS and 20 mg Diastat.

Of the 24 subjects randomized, 20 (83%) were males and 4 (17%) were females. The mean (SD) age was 34.0 (6.77) years and the age range was 21 to 46 years. Slightly more than half of the subjects were White (N=13; 54%); otherwise, subjects were Black or African American (N=8; 33%) and American Indian or Alaska Native (N=3; 13%). In addition, slightly more than half of the subjects were of Hispanic or Latino ethnicity (N=14; 58%) compared to not of Hispanic or Latino ethnicity (N=10; 42%), Subjects' BMI ranged from 26 to 43 kg/m2 (mean [SD]: 31.2 [3.63]).

### Pharmacokinetic Results:

### Plasma Concentration Data—Diazepam

Three subjects had low BA of diazepam (< 1/10 the mean Cₘₐₓ observed in subjects with good BA), following dosing with 20 mg Diastat, but not with either dose of DZNS. Study drug leakage was assessed at 5, 15, 30, 45, and 60 minutes following Diastat administration and some leakage was noted in 7 subjects; however, only the 3 subjects with low BA had leakage noted at the earliest, 5-minute time point. Most PK presentations are, therefore, for both the overall PK Population (not excluding any subjects) and for the PK Population excluding subjects with low BA following administration of 20 mg Diastat. The subset of the PK Population excluding subjects with low BA following administration of 20 mg Diastat was considered to be the most valid group of subjects for comparison with the 20 mg DZNS treatment group, and is the focus of presentations in this study.
Diazepam was rapidly absorbed following administration of each treatment (whether or not subjects with low BA following Diastat administration were included or excluded), with mean peak plasma concentrations occurring at 1 to 1.5 hours after dosing. The highest mean (±SD) plasma concentrations were 96.3±27.7 ng/mL at 1.00 hours for 5 mg DZNS, 350±103 ng/mL at 1.00 hour for 20 mg DZNS, and 352±92.9 ng/mL at 1.50 hours for the reference product (Diastat) (excluding subjects who had low BA following Diastat administration). Following the peak, the concentrations decayed in a bi-phasic manner, with a long terminal phase commencing at about 24 to 48 hours after dosing. Also, it is interesting to note that in ˜50% of the subjects, the observed 48-hour diazepam concentration was slightly higher than the 24-hour concentration, regardless of treatment.

### Plasma Concentration Data—Nordiazepam

Nordiazepam was often measurable prior to dosing at Dosing Periods 2 and 3, and was almost always measurable at 240 hours after dosing of each treatment. The results indicated substantial accumulation between treatments due to exceptionally long half-lives for diazepam in this group of subjects. Therefore, to permit comparison between treatments in the absence of prior diazepam administration, the results presented are for Dosing Period 1. These results indicated that nordiazepam concentrations accumulated very slowly over time, with mean peak plasma concentrations occurring at 96 to 144 hours after dosing. The highest mean (±SD) plasma concentrations were 9.9±3.1 ng/mL at 144 hours for 5 mg DZNS, 37.3±13.0 ng/mL at 96 hours for 20 mg DZNS, and 35.5±14.5 ng/mL at 96 hours for 20 mg Diastat) (excluding subjects who had low BA following Diastat administration). The profiles for mean plasma nordiazepam concentration-time data over 336 hours for Dosing Period 1 (including the pre-dose sample for Dosing Period 2) excluding subjects with low BA following administration of 20 mg Diastat were similar, indicating that there is no route of administration difference in the metabolism of diazepam to nordiazepam.

### Noncompartmental PK Parameters—Diazepam

A summary of noncompartmental PK parameters for diazepam is presented in Table 18. Median Tₘₐₓ values were similar to mean Tₘₐₓ values (1.0 hours after both 5 mg and 20 mg DZNS and 1.25 hours after 20 mg Diastat [excluding subjects with low BA]).
The estimated half-life values for diazepam were long and variable following all treatments. The range of half-lives was from 44.5 to 243 hours (5 mg DZNS), 48.1 to 221 hours (20 mg DZNS), and 43.8 to 234 hours 20 mg Diastat treatment (excluding subjects with low BA). Although the intersubject variability was quite high (52 to 57% CV), the intrasubject variability appeared to be much lower; i.e., PK values within a subject were generally consistent across the three treatment groups.
Because of the long diazepam half-lives, there was considerable AUC extrapolated in the tail in the calculation of AUC_{inf}. Clearance (CL/F) values were similar among the treatments. Vz/F values were large and comparable among the treatments.

TABLE 18
Summary of Noncompartmental PK Parameters for Diazepam: PK Population
(Excluding Subjects with Low BA Following Diastat Administration)
Parameter Mean
[SD]
CV %

| Treatment | Cₘₐₓ | Tₘₐₓ | λz | t½ | | | | CL/F | Vz/F |
|---|---|---|---|---|---|---|---|---|---|
| Group | (ng/mL) | (h) | (1/h) | (h) | AUC₀₋₂₄^{a} | AUCₗₐₛₜ^{a} | AUC_{inf}a | (L/h) | (L) |
| 5 mg | 108 | 0.98 | 0.0089 | 96.2 | 823 | 3205 | 4195 | 1.83 | 201 |
| DZNS | [30.5] | [0.39] | [0.0038] | [50.1] | [285] | [2108] | [3345] | [1.12] | [76.9] |
| (N = 22) | 28.1 | 39.6 | 43.0 | 52.1 | 34.6 | 65.8 | 79.7 | 61.4 | 38.3 |
| 20 mg | 378 | 1.02 | 0.0089 | 98.6 | 2720 | 9860 | 12725 | 2.03 | 240 |
| DZNS | [106] | [0.33] | [0.0040] | [52.6] | [738] | [4419] | [8120] | [0.87] | [82.7] |
| (N = 23) | 28.1 | 32.1 | 44.7 | 53.3 | 27.1 | 44.8 | 63.8 | 42.8 | 34.4 |
| 20 mg | 375 | 1.12 | 0.0085 | 108 | 3015 | 11420 | 14816 | 1.66 | 218 |
| Diastat | [96.8] | [0.45] | [0.0043] | [61.4] | [710] | [4088] | [6967] | [0.80] | [99.0] |
| (N = 20) | 25.9 | 40.1 | 50.3 | 56.8 | 23.6 | 35.8 | 47.0 | 48.2 | 45.3 |

^{a}units for AUC are (h * ng/mL)

### Noncompartmental PK Parameters—Nordiazepam

Nordiazepam noncompartmental PK parameters could only be reliably estimated using Dosing Period 1 results due to the long observed half-lives and continued accumulation during each of the subsequent 2-week study periods. A summary of noncompartmental PK parameters for nordiazepam for Dosing Period 1 is presented in Table 19. The Cₘₐₓ results indicate that the maximum concentrations of nordiazepam were approximately one-tenth those of diazepam, regardless of treatment. Median Tₘₐₓ values were 144 hours after 5 mg DZNS, 96 hours after 20 mg DZNS, and 120 hours after 20 mg Diastat (excluding subjects with low BA). Half-life estimates were extremely long. As a result of the long half-lives for nordiazepam, a significant percentage of the AUC was extrapolated leading to very high AUC_{inf} values.

TABLE 19
Summary of Noncompartmental PK Parameters for Nordiazepam
for Dosing Period 1: PK Population (Excluding Subjects
with Low BA Following Diastat Administration)
Parameter Mean
[SD]
CV %

| Treatment | Cₘₐₓ | Tₘₐₓ | t½ | AUC₀₋₃₃₆ | AUC_{inf} |
|---|---|---|---|---|---|
| Group | (ng/mL) | (h) | (h) | (h*ng/mL) | (h*ng/mL) |
| 5 mg | 10.7 | 141.0 | 235 | 2620 | 4820 |
| DZNS | [3.17] | [81.6] | [140] | [774] | [2119] |
| (N = 8) | 29.5 | 57.9 | 59.7 | 29.5 | 44.0 |
| 20 mg | 39.0 | 126.0 | 237 | 9100 | 16159 |
| DZNS | [11.1] | [50.9] | [239] | [1759] | [10487] |
| (N = 8)^{a} | 28.5 | 40.4 | 100.9 | 19.3 | 64.9 |
| 20 mg | 38.9 | 160.1 | 294 | 9138 | 20125 |
| Diastat | [12.2] | [108] | [293] | [2586] | [13112] |
| (N = 6)^{b} | 31.3 | 67.5 | 99.5 | 28.3 | 65.2 |

^{a}N = 7 for t½ and AUC_{inf}
^{b}N = 5 for t½ and AUC_{inf}

In total, the results indicate that there is no route of administration difference for the formation of nordiazepam between IN and rectal administration.

### Comparative BA Analysis

As presented above (Plasma Concentration Data—Diazepam), 3 subjects displayed very low plasma diazepam concentrations following administration of 20 mg Diastat, and so a subset analysis of relative BA using the two one-sided test procedure was conducted using the subjects with good BA, in addition to the complete PK Population. Exclusion of subjects with low BA following Diastat administration was based on a review of the distribution of the Cₘₐₓ and AUC values.
When the 3 subjects with low BA following administration of Diastat are included in the analysis, the test formulation has a ratio exceeding 100% for Cₘₐₓ, AUC₀₋₂₄, AUCₗₐₛₜ, and AUC_{inf}, and the 90% CI for the ratio is outside of the 80% to 125% acceptance interval. This result is likely due to the influence of the 3 outliers not only on the ratio, but in the distribution of the data. In contrast, when the 3 subjects with low BA following administration of Diastat are excluded from the analysis, the 90% CIs are within the 80 to 125% acceptance interval for Cₘₐₓ (85.30, 113.64) and AUC₀₋₂₄ (80.23, 97.72), and slightly outside for AUCₗₐₛₜ (75.44, 94.42) and AUC_{inf} (75.34, 91.68).

### Dose Proportionality Analysis

Due to the observed long half-lives of diazepam, which ranged from 44.5 to 243 hours across subjects and treatments, there was some carry-over for diazepam, especially for subjects with long diazepam half-lives, i.e., those exceeding 80 to 100 hours. This carry over was of greatest importance when a 5 mg DZNS treatment followed a 20 mg DZNS or 20 mg Diastat treatment. Therefore, it was necessary to correct the data for the dose proportionality assessment by subtracting the residual diazepam from a prior dose from the measured concentrations over time foreach subject by using the average terminal phase rate constant value for that subject.

The results of the two one-sided test indicated that the 90% CI for Cₘₐₓ, AUC₀₋₂₄, AUC_{inf}, and CL/F were all within the 80 to 125% standard equivalence interval, indicating that the 5 mg DZNS treatment showed dose proportionality to the 20 mg DZNS treatment.

### Safety Results:

Twenty-one subjects (96%), 23 subjects (100%), and 17 subjects (74%) reported at least one TEAE in the 5 mg DZNS, 20 mg DZNS, and 20 mg Diastat treatment groups, respectively, and the same number and percentage of subjects in each treatment group reported at least one treatment-related TEAE, All TEAEs were mild or moderate in intensity. There were no SAEs and no TEAEs that led to discontinuation.
Most TEAEs reflected abnormalities of one of three system organ classes: Eye Disorders; Nervous System Disorders; or Respiratory, Thoracic and Mediastinal Disorders. The most common TEAE was lacrimation increased, reported about equally in the two IN dose groups (82% and 78% of subjects in the 5 mg and 20 mg DZNS treatment groups, respectively), compared to no subjects in the 20 mg Diastat treatment group. This TEAE typically occurred immediately or within minutes of dosing, was always mild, and was of short duration (≦3 hours). The second most common TEAE was somnolence. Somnolence appeared to be dose-related; it was reported with similar frequencies in the 20 mg DZNS and 20 mg Diastat treatment groups (52% and 61% incidence, respectively), compared with 23% of subjects reporting this TEAE in the 5 mg DZNS treatment group. Other common TEAEs (rhinorrhoea, nasal inflammation, nasal congestion, and nasal discomfort) likely reflected local effects or else were likely systemic TEAEs that appeared to be dose-related (i.e., dizziness, reported with similar frequency in the 20 mg DZNS and 20 mg Diastat treatment groups [17% and 22%, respectively] compared to 5% in the 5 mg DZNS treatment group.

No clinically significant observations or changes in other safety parameters were identified in the subject population during the study conduct based on results of physical examinations, clinical laboratory assessments, or ECUs. There were no positive C-SSRS findings.

There were no clinically significant changes in pulse oximetry, HR, respiratory rate, or temperature following dosing in any of the three treatment groups. Furthermore, there were no clinically significant changes in SBP, DBP, or HR following IN dosing with either 5 mg DZNS or 20 mg DZNS. However, following rectal administration of 20 mg Diastat, SBP and DBP (but not HR) each decreased by a mean of about 15 to 17 mmHg at the 15 and 30 minute post-dosing time points, returning to pre-dose values at 1 hour post-dose, the next time point assessed. This pattern was also observed in the 3 subjects with low BA following administration of Diastat. These drops in blood pressure after dosing with 20 mg Diastat were usually not associated with symptoms.
For the Nasal and Pharyngeal Irritation/Inflammation Assessment, nasal signs or symptoms, usually signs of nasal redness, congestion or runny nose, were seen most frequently in the 5 mg DZNS treatment group at 0.5 hours post-dose (in 7 of 23 subjects [32%]) and were seen most frequently in the 20 mg DZNS treatment group at 1 hour post-dose (in 10 of 23 subjects [48%]). Nasal signs and symptoms were resolved for most subjects by 8 hours post-dose (reported by 0 subjects in the 5 mg DZNS treatment group and 3 of 23 subjects [13%] in the mg DZNS treatment group). These frequencies were similar to, or less than, pre dose percentages. Similarly, the percentages of subjects with signs or symptoms in the nasal cavity at 24 hours post-dose were similar to, or less than, pre-dose results (1 of 22 subjects [5%] in the 5 mg DZNS treatment group and 1 of 23 subjects [4%] in the 20 mg DZNS treatment group). Pharyngeal signs or symptoms were less common; they were never reported by more than 2 of 23 subjects (9%) in either DZNS treatment group at any time point. There were no subjects with pharyngeal signs or symptoms at 24 hours post dose.
For the Subject Alertness Observation, more subjects were alert at more post-dose time points up to 4 hours post dose in the 5 mg DZNS treatment group (82 to 100%) compared to the 20 mg DZNS (35 to 87%) and 20 mg Diastat (44 to 96%) treatment groups. After all three treatments, the time point with the fewest alert subjects was 1 hour post-dose (82%, 35%, and 44% of subjects in the three treatment groups, respectively). At 1 hour post-dose, nonalert subjects were primarily drowsy if they had been given either dose of DZNS (18%, 39%, and 13% in the 5 mg DZNS, 20 mg DZNS, and 20 mg Diastat treatment groups, respectively); however, if they had been given 20 mg Diastat, non-alert subjects were primarily sleeping but arousable (0, 26%, and 44% in the 5 mg DZNS, 20 mg DZNS, and 20 mg Diastat treatment groups, respectively). By 2 hours post-dose, ≧75% of subjects in all three treatment groups were alert, except for 4 hours post-dose in the 20 mg DZNS treatment group (70% were alert). At 4 hours after dosing, 5%, 22%, and 4% of subjects in the 5 mg DZNS, 20 mg DZNS, and 20 mg Diastat treatment groups, respectively, were drowsy and 0, 9% and 4% of subjects in the respective treatment groups were sleeping but arousable. All subjects were alert at 24 hours post-dose, and no subject at any time during the study was identified as sleeping but not arousable.

### Conclusions:

### Pharmacokinetic

The results of this study indicated that the BA, as evidenced by the rate and extent of absorption of diazepam from the IN 20 mg DZNS dose, is comparable to that of 20 mg Diastat, administered rectally.
The PK of IN diazepam doses of 5 mg and 20 mg DZNS are proportional with respect to Cmax and AUC.
There was no route of administration difference observed in the metabolism of diazepam to nordiazepam following IN versus rectal administration.

### Safety

All doses and formulations (5 mg and 20 mg DZNS and 20 mg Diastat) were well tolerated with safety profiles as expected.
The safety profile of the test products (5 mg and 20 mg DZNS) was similar to the reference product (Diastat), with the exception that local, transient, and always mild nasal/pharyngeal TEAEs and other adverse nasal/pharyngeal observations were more frequently observed following DZNS administration, as compared to Diastat administration. In addition, systemic TEAEs, such as somnolence and dizziness, and observations of decreased alertness were more common following administration of the two 20 mg dose formulations (20 mg DZNS and 20 mg Diastat) compared to administration of 5 mg DZNS.

### Example 8

A GLP toxicity study of an intranasal 2.5% diazepam formulation (below) in rabbits was performed.

### 2.5% Intranasal Dose Formulation

| Ingredient (Trade Name) | % wt/wt |
|---|---|
| Diazepam | 2.50 |
| Diethylene glycol monoethyl ether, NF (Transcutol ® HP) | 48.20 |
| Propylene glycol monocaprylate (Capryol ™ 90) | 7.60 |
| Methyl laurate | 9.50 |
| N-methyl-2-pyrrolidone (Pharmasolve ®) | 22.70 |
| Ethanol, NF | 7.60 |
| Purified Water, USP | 1.90 |

Rabbits tolerated intranasal administration of a 50 μL dose of the formulation three times weekly for 26 weeks, which delivered approximately 1.25 mg of diazepam/dose. This was considered to be the maximum feasible dose volume, and it meant that the rabbits received approximately the same volume per surface area as patients would receive at the recommended therapeutic dose. The only effect of chronic administration was minimal local irritation at the site of administration in the nasal cavity and sinuses, which resolved when dosing stopped.

The foregoing is illustrative of the present invention, and is not to be construed as limiting thereof. The invention is defined by the following claims, with equivalents of the claims to be included therein. All publications, patent applications, patents, patent publications, and other references cited herein are incorporated by reference in their entireties for the teachings relevant to the sentence and/or paragraph in which the reference is presented.

That which is claimed is:

1. A pharmaceutical composition comprising about 1% to about 15% by weight of a benzodiazepine or a pharmaceutically acceptable salt thereof, about 43% to about 55% by weight of a glycol ether, about 16% to about 18% by weight one or more fatty acid esters, about 22% to about 25% by weight N-methyl-2-pyrrolidone, about 1% to about 5% by weight water, and about 5% to about 10% by weight ethanol.

2. The pharmaceutical composition of claim 1, comprising about 1% to about 15% by weight diazepam or a pharmaceutically acceptable salt thereof, about 43% to about 55% by weight diethylene glycol monoethyl ether, about 9% to about 10% by weight methyl laurate, about 7% to about 9% by weight propylene glycol monocaprylate, about 22% to about 25% by weight N-methyl-2-pyrrolidone, about 1% to about 5% by weight water, and about 5% to about 10% by weight ethanol.

3. The pharmaceutical composition of claim 1, comprising 2.50% by weight diazepam or a pharmaceutically acceptable salt thereof, 48.20% by weight diethylene glycol monoethyl ether, 7.60% by weight propylene glycol monocaprylate, 9.50% by weight methyl laurate, 22.70% by weight N-methyl-2-pyrrolidone, 7.60% by weight ethanol, and 1.90% by weight water.

4. The pharmaceutical composition of claim 1, comprising 3.75% by weight diazepam or a pharmaceutically acceptable salt thereof, 46.95% by weight diethylene glycol monoethyl ether, 7.60% by weight propylene glycol monocaprylate, 9.50% by weight methyl laurate, 22.70% by weight N-methyl-2-pyrrolidone, 7.60% by weight ethanol, and 1.90% by weight water.

5. The pharmaceutical composition of claim 1, comprising 5.00% by weight diazepam or a pharmaceutically acceptable salt thereof, 45.70% by weight diethylene glycol monoethyl ether, 7.60% by weight propylene glycol monocaprylate, 9.50% by weight methyl laurate, 22.70% by weight N-methyl-2-pyrrolidone, 7.60% by weight ethanol, and 1.90% by weight water.

6. The pharmaceutical composition of claim 1, comprising 6.25% by weight diazepam or a pharmaceutically acceptable salt thereof, 44.45% by weight diethylene glycol monoethyl ether, 7.60% by weight propylene glycol monocaprylate, 9.50% by weight methyl laurate, 22.70% by weight N-methyl-2-pyrrolidone, 7.60% by weight ethanol, and 1.90% by weight water.

7. The pharmaceutical composition of claim 1, comprising 7.50% by weight diazepam or a pharmaceutically acceptable salt thereof, 43.20% by weight diethylene glycol monoethyl ether, 7.60% by weight propylene glycol monocaprylate, 9.50% by weight methyl laurate, 22.70% by weight N-methyl-2-pyrrolidone, 7.60% by weight ethanol, and 1.90% by weight water.

8. The pharmaceutical composition of claim 1, comprising 8.75% by weight diazepam or a pharmaceutically acceptable salt thereof, 41.95% by weight diethylene glycol monoethyl ether, 7.60% by weight propylene glycol monocaprylate, 9.50% by weight methyl laurate, 22.70% by weight N-methyl-2-pyrrolidone, 7.60% by weight ethanol, and 1.90% by weight water.

9. The pharmaceutical composition of claim 1, comprising 10.00% by weight diazepam or a pharmaceutically acceptable salt thereof, 40.70% by weight diethylene glycol monoethyl ether, 7.60% by weight propylene glycol monocaprylate, 9.50% by weight methyl laurate, 22.70% by weight N-methyl-2-pyrrolidone, 7.60% by weight ethanol, and 1.90% by weight water.

10. A pharmaceutical composition comprising about 1% to about 10% by weight of a benzodiazepine or a pharmaceutically acceptable salt thereof, about 40% to about 47% by weight of a glycol ether, and about 45% to about 55% by weight one or more fatty acid esters.

11. The pharmaceutical composition of claim 10, further comprising about 0.5% to about 3% by weight water.

12. The pharmaceutical composition of claim 10, comprising about 1% to about 10% by weight diazepam or a pharmaceutically acceptable salt thereof, about 40% to about 47% by weight diethylene glycol monoethyl ether, about 26% to about 34% by weight caprylocaproyl polyoxylglyceride, about 5% to about 10% by weight oleoyl polyoxylglyceride, and about 5% to about 15% by weight sorbitan monolaurate 20.

13. The pharmaceutical composition of claim 10, comprising 4.95% by weight diazepam or a pharmaceutically acceptable salt thereof, 45.62% by weight diethylene glycol monoethyl ether, 30.42% by weight caprylocaproyl polyoxylglyceride, 7.6% by weight oleoyl polyoxylglyceride, and 11.41% by weight sorbitan monolaurate 20.

14. The pharmaceutical composition of claim 10, comprising 6.63% by weight diazepam or a pharmaceutically acceptable salt thereof, 44.82% by weight diethylene glycol monoethyl ether, 29.88% by weight caprylocaproyl polyoxylglyceride, 7.47% by weight oleoyl polyoxylglyceride, and 11.20% by weight sorbitan monolaurate 20.

15. The pharmaceutical composition of claim 11, comprising about 1% to about 10% by weight diazepam or a pharmaceutically acceptable salt thereof, about 40% to about 47% by weight diethylene glycol monoethyl ether, about 26% to about 34% by weight caprylocaproyl polyoxylglyceride, about 5% to about 10% by weight isopropyl palmitate, about 5% to about 15% by weight sorbitan monolaurate 20, and about 0.5% to about 3% by weight water.

16. The pharmaceutical composition of claim 11, comprising 2.50% by weight diazepam or a pharmaceutically acceptable salt thereof, 48.10% by weight diethylene glycol monoethyl ether, 7.30% by weight isopropyl palmitate, 10.80% by weight sorbitan monolaurate 20, 30.30% by weight caprylocaproyl polyoxylglyceride, and 1.00% by weight water.

17. The pharmaceutical composition of claim 11, comprising 3.75% by weight diazepam or a pharmaceutically acceptable salt thereof, 46.85% by weight diethylene glycol monoethyl ether, 7.30% by weight isopropyl palmitate, 10.80% by weight sorbitan monolaurate 20, 30.30% by weight caprylocaproyl polyoxylglyceride, and 1.00% by weight water.

18. The pharmaceutical composition of claim 11, comprising 5.0% by weight diazepam or a pharmaceutically acceptable salt thereof, 45.60% by weight diethylene glycol monoethyl ether, 7.30% by weight isopropyl palmitate, 10.80% by weight sorbitan monolaurate 20, 30.30% by weight caprylocaproyl polyoxylglyceride, and 1.00% by weight water.

19. The pharmaceutical composition of claim 11, comprising 5.0% by weight diazepam or a pharmaceutically acceptable salt thereof, 45.60% by weight diethylene glycol monoethyl ether, 7.22% by weight isopropyl palmitate, 10.83% by weight sorbitan monolaurate 20, 30.40% by weight caprylocaproyl polyoxylglyceride, and 0.95% by weight water.

20. The pharmaceutical composition of claim 11, comprising 6.25% by weight diazepam or a pharmaceutically acceptable salt thereof, 44.35% by weight diethylene glycol monoethyl ether, 7.30% by weight isopropyl palmitate, 10.80% by weight sorbitan monolaurate 20, 30.30% by weight caprylocaproyl polyoxylglyceride, and 1.00% by weight water.

21. The pharmaceutical composition of claim 11, comprising 7.50% by weight diazepam or a pharmaceutically acceptable salt thereof, 43.10% by weight diethylene glycol monoethyl ether, 7.30% by weight isopropyl palmitate, 10.80% by weight sorbitan monolaurate 20, 30.30% by weight caprylocaproyl polyoxylglyceride, and 1.00% by weight water.

22. The pharmaceutical composition of claim 11, comprising 8.75% by weight diazepam or a pharmaceutically acceptable salt thereof, 41.85% by weight diethylene glycol monoethyl ether, 7.30% by weight isopropyl palmitate, 10.80% by weight sorbitan monolaurate 20, 30.30% by weight caprylocaproyl polyoxylglyceride, and 1.00% by weight water.

23. The pharmaceutical composition of claim 11, comprising 10.00% by weight diazepam or a pharmaceutically acceptable salt thereof, 40.60% by weight diethylene glycol monoethyl ether, 7.30% by weight isopropyl palmitate, 10.80% by weight sorbitan monolaurate 20, 30.30% by weight caprylocaproyl polyoxylglyceride, and 1.00% by weight water.

24. The pharmaceutical composition of claim 1, wherein the benzodiazepine is diazepam.

25. The pharmaceutical composition of claim 1, wherein the glycol ether is diethylene glycol monoethyl ether.

26. The pharmaceutical composition of claim 1, wherein the one or more fatty acid esters is selected from the group consisting of caprylocaproyl polyoxylglyceride, isopropyl palmitate, oleoyl polyoxylglyceride, sorbitan monolaurate 20, methyl laurate, ethyl laurate, polysorbate 20, propylene glycol monocaprylate, and any combination thereof.

27. The pharmaceutical composition of claim 1, wherein the one or more fatty acid esters is selected from the group consisting of methyl laurate, propylene glycol monocaprylate, and any combination thereof.

28. The pharmaceutical composition of claim 10, wherein the one or more fatty acid esters is selected from the group consisting of caprylocaproyl polyoxylglyceride, isopropyl palmitate, sorbitan monolaurate 20, and any combination thereof.

29. The pharmaceutical composition of claim 10, wherein the one or more fatty acid esters is selected from the group consisting of caprylocaproyl polyoxylglyceride, oleoyl polyoxylglyceride, sorbitan monolaurate 20, and any combination thereof.

30. The pharmaceutical composition of claim 1, in a form for intranasal administration.

31. A pharmaceutical composition for intranasal administration of diazepam, comprising diazepam or a pharmaceutically acceptable salt thereof, a glycol ether, and one or more fatty acid esters, wherein upon administration to a human subject, plasma levels of diazepam exhibit a coefficient of variation (CV) of less than about 40%.

32. The pharmaceutical composition of claim 31, wherein the CV is less than about 30%.

33. An intranasal spray device comprising the pharmaceutical composition of claim 1.

34. A method of treating a seizure in a subject, comprising intranasally administering a therapeutically effective amount of the pharmaceutical composition of claim 1 to a subject in need thereof.

35. The method of claim 34, wherein after administration of the composition to a subject, the subject's blood pressure is maintained at a consistent level for at least 1 hour.

36. The method of claim 35, wherein the subject's blood pressure after administration of the composition remains within 10/10 mmHg (SBP/DBP) of the subject's blood pressure prior to administration of the composition.

37. The method of claim 34, wherein after administration of the composition to a subject, the subject's pulse is maintained at a consistent level for at least one hour.

38. The method of claim 37, wherein the subject's pulse remains within 5 beats per minute of the subject's pulse prior to administration of the composition.

39. A method of preventing a drop in blood pressure in a subject during administration of diazepam for treatment of a seizure, comprising intranasally administering a therapeutically effective amount of the pharmaceutical composition of claim 1 to a subject in need thereof.
